(19) United States
(12) Patent Application Publication
(10) Pub. No.: US 2016/0317648 A1
(43) Pub. Date: Nov. 3, 2016

(54) CAP260, CAP174 AND K0224 HIV-1 ENVELOPES, PEPTIDE AND COMPOSITIONS

(71) Applicants: DUKE UNIVERSITY, Durham, NC (US); LOS ALAMOS NATIONAL SECURITY, LLC, Los Alamos, NM (US)

(72) Inventors: Barton F. HAYNES, Durham, NC (US); Bette T. KORBER, Los Alamos, NM (US)

(21) Appl. No.: 15/105,214

(22) PCT Filed: Dec. 18, 2014
(86) PCT No.: PCT/US2014/071117

§ 371 Date: Jun. 16, 2016

Related U.S. Application Data
(60) Provisional application No. 61/917,561, filed on Dec. 18, 2013.
(60) Provisional application No. 61/940,987, filed on Feb. 18, 2014.

(51) Int. Cl. A61K 39/21; C12N 7/00; A61K 39/39; C07K 14/005

(52) U.S. Cl. CPC A61K 39/21; C07K 14/005; C12N 7/00; A61K 39/39; A61K 2039/575

(57) ABSTRACT

The invention is directed to HIV-1 envelope proteins and peptides, and compositions comprising the same to increase the breadth of vaccine coverage of the V1 V2 env region of clade C HIV-1.

This application claims the benefit of U.S. Application Ser. No. 61/917,561 filed Dec. 18, 2013 and U.S. Application Ser. No. 61/940,987 filed Feb. 18, 2014, the entire content of each application is herein incorporated by reference.

This invention was made with government support under Grant No. AI 067854 awarded by the National Institutes of Health. The government has certain rights in the invention.

### TECHNICAL FIELD

The present invention relates in general, to a composition suitable for use in inducing anti-HIV-1 antibodies, and, in particular, to immunogenic compositions comprising peptides and envelope proteins to increase the breadth of coverage of the V1V2 envelope region of clade C in a candidate HIV-1 virus vaccine. The invention also relates to methods of inducing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

One of the major challenges to HIV-1 vaccine development has been the inability of immunogens to induce broadly neutralizing antibodies (nAb). nAbs are generated during HIV-1 infection. However, most of the nAbs generated neutralize only the autologous viruses or closely related strains (Moog et al, J. Virol. 71:3734-3741 (1997), Gray et al, J. Virol. 81:6187-6196 (2007)). HIV envelope (Env) constantly mutates to escape from existing nAb response (Albert et al, Aids 4:107-112 (1990), Wei et al, Nature 422:307-312) (2003)). nAb responses do evolve over the course of the HIV infection. However, with the mutation capacity of HIV-1 viruses, neutralizing antibody responses always seem to “lag behind” virus evolution (Wei et al, Nature 422:307-312 (2003)), Richman et al, Proc. Natl. Acad. Sci. USA 100:4144-4149 (2003), Geffin et al, Virology 310:207-215 (2003)). Moreover, Rolland et al. Nature 490: 417-20, 2012 found that when the infecting virus in the cohort of vaccinees in the RV144 ALVAC/AIDSVAX B/E trial had a K at the second variable loop (V2) amino acid position 169, there was 48% efficacy and when there was no K at 169 there was no efficacy. Thus, expanding the immunogens to induce antibodies against other amino acids in the V2 region around aa 169 should increase vaccine efficacy. A selection of multiplicity of immunogens would provide greater coverage of an immunogenic region such as the V2 HIV Env region and is expected to increase the breadth of the immune response elicited by these immunogens.

### SUMMARY OF THE INVENTION

The invention provides peptides, envelope proteins and compositions comprising the same to increase the breadth of coverage of the V1V2 env region of clade C in a candidate HIV-1 virus vaccine used in South African efficacy trial. See Ruffin et al. Journal of Translational Medicine 2012, 10:144, at page 2. In certain embodiments, the invention provides compositions comprising polyvalent immunogens to increase the breadth of coverage of the V1V2 env region of clade C in a candidate HIV-1 virus vaccine used in South African efficacy trial. In certain embodiments the invention provides a pentavalent Subtype C composition comprising CAP260, CAP174 or Ko224 HIV-1 Envelopes or peptides. Provided are methods to use three, CAP260, CAP174 or Ko224 HIV-1 Envelopes or peptides as addition to the current vaccine components that would increase the coverage of ADCC in induced by TV1 and 1086C.

In certain aspects, the invention provides a synthetic peptide comprising, consisting essentially of, or consisting of sequences as described in FIG. 2, FIG. 13, or Example 1, or a combination thereof. In certain aspects, the peptide includes a tag. In certain embodiments, the tag is biotin.

In certain aspects, the invention provides a CAP260, CAP174 or Ko224 HIV-1 Envelopes, or a combination thereof. In certain embodiments, the proteins are recombinant. In certain embodiments, these are CAP260, CAP174 or Ko224 proteins of FIG. 6, FIG. 11, FIG. 12, or a combination thereof. In certain embodiments, the proteins are gp120 proteins. In certain embodiments, the proteins are gp160 proteins. In certain embodiments, the proteins are gp140 protein. In certain embodiments, the gp140 protein is a gp140C, gp140CF, gp140CFI. See US Pub 2012/0321699, US Pub 20112/0087938, and US Pub 2012/0183597 incorporated by reference. In certain embodiments, the proteins comprise sequence modifications making them less resistant to protein cleavage during recombinant production in CHO cells. In other embodiments the invention provides variants of the CAP260, CAP174 or Ko224 HIV-1 Envelopes sequences, wherein the variants comprise a mutation which repairs a tripsin cleavage site, thereby preventing protein clipping during envelope protein production in a cell line, e.g., a CHO cell line. In certain embodiments, the proteins comprise the following V3 loop sequence TRPNNNTRKSIRIGPGQTFYATGDIIGNIRQAH. See U.S. Ser. No. 61/807,644 which entire content is hereby incorporated by reference. In certain embodiments, the envelope protein is present essentially as monomer. In certain embodiments, the monomer is at least 80%, at least 85% at least 90%, at least 91%, at least 92%, at least 93%, at least 94%, at least 95%, at least 96%, at least 97%, at least 98%, at least 99% of the envelope in a composition.

In certain aspects, the invention provides nucleic acids encoding the inventive envelope proteins, for example of FIG. 6, FIG. 11, or FIG. 12. In certain aspects, the invention provides vectors comprising these nucleic acids. In certain aspects, the invention provides codon optimized nucleic acid sequence encoding the inventive envelope proteins, for example of FIG. 6, FIG. 11, or FIG. 12. In certain embodiments the vectors are suitable for use in a vaccine formulation. In certain embodiments the vector is A pox virus vector (for example but not limited to ALVAC), a vaccinia vector (for example but not limited to NYVAC).

In certain aspects, the invention provides a composition comprising, consisting essentially of, consisting of a synthetic peptide of FIG. 2, FIG. 13, Example 1, or a combination thereof. In certain embodiments, the invention provides a composition comprising, consisting essentially of, consisting of a trivalent combination of CAP260, CAP174 and Ko224 peptides, for example as described in FIG. 2, FIG. 13, Example 1. In certain embodiments, the invention provides a composition comprising/consisting essentially of/consisting of a trivalent combination of CAP260, CAP174 and Ko224 envelopes, or nucleic acids encoding these. In certain embodiments, the composition comprises an adjuvant. In certain embodiments, the inventive reagents and compositions are immunogenic.

In certain aspects the invention provides a composition comprising peptides of SEQ ID NOs: 8, 9 and 10, SEQ ID NOs: 29, 30 and 31, or Example 1 (SEQ ID NO: 64, 68, and 72), or FIG. 13, or a combination thereof.

In certain aspects the invention provides a composition comprising recombinant CAP260, CAP174 and Ko224 HIV-1 Envelope proteins of FIG. 6 (SEQ ID NOs: 55-60) as gp120, or gp160 proteins, or nucleic acids encoding these.

In certain embodiments of the compositions the protein is present essentially as a monomer. In certain embodiments of the compositions the protein is present at least 80%, at least 85% at least 90%, at least 91%, at least 92%, at least 93%, at least 94%, at least 95%, at least 96%, at least 97%, at least 98%, or at least 99% as a monomer.

In certain embodiments, the compositions further comprise Env gp120 TV1 and Env gp120 1086C.

In certain aspects, the invention provides a method of inducing antibodies in a subject comprising administering to the subject any of the inventive compositions described herein, in any suitable manner. In certain aspects, the invention provides a method of inducing antibodies in a subject comprising administering to the subject a composition comprising a trivalent combination of CAP260, CAP174 and Ko224 peptides, or CAP260, CAP174 and Ko224 envelopes, or nucleic acids encoding these. In certain embodiments, the composition is administered as a prime. In certain embodiments, the composition is administered as a boost in an immunization regimen following administration of ZM-96gp120 as a prime, and TV-1 and 1086C gp120 envelope proteins as a boost. In certain embodiments, the composition is administered as a multiple boost. In certain embodiments, the inventive compositions, alone or in combination with the current South African vaccine components and immunization regimens, induce broadly neutralizing antibodies.

In certain aspects the invention provides methods of inducing antibodies in a subject comprising administering to the subject any one of the compositions, for example but not limited to a composition comprising a trivalent combination of CAP260, CAP174 and Ko224 peptides, CAP260, CAP174 and Ko224 recombinant proteins, or DNA vectors encoding CAP260, CAP174 and Ko224 proteins.

In certain embodiments, these compositions are administered as a prime in an immunization regimen of ZM-96gp120 as a prime, and TV-1 and 1086C gp120 envelope proteins as a boost. In certain embodiments, these compositions are administered as a boost in an immunization regimen following administration of ZM-96gp120 as a prime, and TV-1 and 1086C gp120 envelope proteins as a boost. In certain embodiments, these compositions are administered as a multiple boost.

In certain embodiments, wherein the composition comprises a trivalent combination of CAP260, CAP174 and Ko224 peptides, CAP260, CAP174 and Ko224 recombinant proteins, or DNA vectors encoding CAP260, CAP174 and Ko224 proteins, the composition is administered as a prime in an immunization regimen along with administration of ZM-96gp120 as a prime, and TV-1 and 1086C gp120 envelope proteins as a boost.

In certain embodiments, the compositions comprise a pentavalent combination of CAP260, CAP174 and Ko224 peptides of claim 1, CAP260, CAP174 and Ko224 recombinant proteins, or DNA vectors encoding CAP260, CAP174 and Ko224 proteins, and Env gp120s (TV1 and 1086C), wherein the pentavalent combination is administered as a boost in an immunization regimen after administration of ZM-96gp120 as a prime.

In certain aspect the invention provides reagents and compositions for use as additional immunogens in the South African trial. In non-limiting embodiments, the immunogen would be either a trivalent V1V2 peptide mixture (Cys 157 modified or unmodified), a C4-V2 trivalent peptide mixture or a trivalent Env mixture, for example but not limited to gp120 proteins (SEQ ID NOs: 56, 58, 60), envelopes in FIG. 11, FIG. 12, or nucleic acids encoding envelopes as described herein. In some embodiments, these compositions further comprise TV-1 and 1086C gp120 envelope protein. Immunogenicity studies can be done in any suitable animal model, for example NHPs.

### BRIEF DESCRIPTION OF THE DRAWINGS

FIG. 1 shows the V1V2 hypervariable regions of TV1, 96ZM651, 1086C which are the immunogens used in the South African vaccine. The vaccine versions of these regions are shown in this figure—the sequence of all three regions are contiguous. SEQ ID NO: 1, 2, and 3 are shown in order of appearance. In the figure, for purpose of illustration, the regions of interest are delineated by tabs. SEQ ID NO: 73, 74, and 75 correspond to the V1V2 epitope of the peptide of SEQ ID NOs: 1, 2 and 3.

FIG. 2A shows sequence analyses and sequences for use as immunogens. Red/underlined amino acids in and under the Cacute_CONSENSUS are common C clade variants that are missing from the initial vaccine set. Red/underlined amino acids in the sequences themselves indicate amino acids not covered by the vaccines. A dash in a sequence indicates that the amino acid at that position is identical to the amino acid sequence in the Cacute_CONSENSUS sequence. Cacute_CONSENSUS is SEQ ID NO: 4. The rest of the sequences in order of appearance correspond to SEQ ID NOs: 5-24 (e.g. C-TV1 is SEQ ID NO: 5, and so forth). In certain embodiments the Cysteine at position 157 is changed to Serine: e.g. Cacute_CONSENSUS Cys to Ser (italicized) peptide has the following sequence EMKNSSFNTTTELRDKKQKEYALFYRLDIVPL_ (SEQ ID NO: 25). Cys to Ser versions of the peptides in this figure correspond to SEQ ID NOs: 25-45. In certain embodiments, the peptides are modified as to allow their tethering and use in an immunoassay. In certain embodiments, the peptides are biotinylated and can be used in an ELISA assay.

FIG. 2B shows the envelope sequences used in the RV144 trial (SEQ ID NOs: 46-54).

FIG. 3 shows the distribution of V1+V2 lengths in the HIV database of C subtype sequences, including only 1 per person (some of whom are likely to be acute or early), versus sequence known to be derived from acute infection and TF or highly similar to TF viruses. The vaccine sequences are shown for comparison as nicks along the bottom, green (names below the X-axis) are new boost suggestions, blue (names above the x-axis) are the current vaccine sequences.

FIG. 4 shows the net charge distribution among C acutes and the C database. The figure shows that there is a slight shift up at transmission, not significant, and the majority of sequences are negatively charged overall. As positively hypervariable loops are clearly important for PG9 like antibody sensitivity, the goal is to identify sequences with positive charge.

FIG. 5 shows the coverage of different populations by different vaccine sets (three original versus three original+three in a boost) are also indicated in a LOGO plot. The red amino acids on the left in the LOGOS are amino acids not captured in the original three vaccine set. The LOGO on the right shows what is gained in terms of vaccine coverage by adding three more sequences as described herein.

FIG. 6 show the envelope sequences of CAP260 (SEQ ID NOs: 55 and 56), CAP174 (SEQ ID NOs: 57 and 58), and Ko224 (SEQ ID NOs: 59 and 60).

FIG. 7 shows HIV-1 C clade phylogeny (maximum likelihood built with FastTree). The tree shows where the vaccines are distributed relative to the C clade globally.

FIG. 8 shows a phylogenetic tree that compares S. African C clade isolates from the database (N=623 individuals) to Thai CRF01 isolates (N=245 individuals), with the vaccine strains indicated by colored boxes. The figure shows the differences in the divergence in the two populations.

FIG. 9 shows that short positively charged loops are associated with increased Tier 2 Env sensitivity to bNABs.

FIG. 10 shows that short positively charged loops are associated with increased Tier 2 Env sensitivity to bNABs.

FIG. 11 shows sequences for use as immunogens (SEQ ID NOs: 76-81 in order of appearance). Red/underlined amino acids indicate a V1V2 peptide that can be used as an immunogen. Italicized are amino acids which are deleted in the delta 11 gp120 design.

FIG. 12 shows amino acid and nucleic acid sequences of a Delta11 gp120 design for use as immunogens (SEQ ID NOs: 82-87 in order of appearance). Task 1: Gene synthesis of 3 genes including HV1300500, HV1300501 and HV1300502 per sequences shown below. Task 2: Subclone the synthesized 3 genes including HV1300500, HV1300501 and HV1300502 into plasmid pcDNA3.1+/Hygromycin at NheI-XbaI sites.

FIG. 13 shows peptide designs (SEQ ID NOs: 88-110).

FIG. 14 shows that immunization of guinea pigs with the immunogens on the X axis induces antibodies in plasma that block the binding of the HIV Env broadly neutralizing antibody (bnAb), CH01 to the A244 gp120 envelope that was an immunogen in the RV144 vaccine efficacy trial. CH01 is a bnAb that binds to glycans at N156, N160 and as well to the K at 169.

FIG. 15 shows that immunization of guinea pigs with the immunogens on the X axis induces antibodies in plasma that block the binding of the HIV V2 antibody CH58 isolated from vaccinees in the RV144 vaccine efficacy trial. CH58 is an antibody that mediates ADCC that binds to Env at the K at 169.

FIG. 16 shows that immunization of guinea pigs with the immunogens on the X axis induces antibodies in plasma that block the binding of the HIV V2 antibody CH59 isolated from vaccinees in the RV144 vaccine efficacy trial. CH59 is an antibody that mediates ADCC that binds to Env at the K at 169.

FIG. 17 shows that immunization of guinea pigs with the immunogens on the X axis induces antibodies in plasma that block the binding of the HIV Env broadly neutralizing antibody (bnAb), PG9 to the A244 gp120 envelope that was an immunogen in the RV144 vaccine efficacy trial. PG9 is a bnAb that binds to glycans at N156, N160 and as well to the K at 169.

FIG. 18 shows that immunization of guinea pigs with the immunogens on the X axis induces antibodies in plasma that block the binding of the HIV Env broadly neutralizing antibody (bnAb), PGT125 to the A244 gp120 envelope that was an immunogen in the RV144 vaccine efficacy trial. PGT 125 is a bnAb that binds to glycans at N332 and as well to amino acids at the base of the V3 loop of HIV Env.

FIG. 19 shows that immunization of guinea pigs with the immunogens on the X axis induces antibodies in plasma that block the binding of soluble CD4 to the CD4 binding site on the clade C transmitted/founder Env CHSO5 gp120. These data show that the immunogens on the X axis are inducing CD4 binding site antibodies.

FIG. 20 shows that the immunogens on the right side of the slide portrayed with the symbols induce the titers of neutralizing antibodies shown as Inhibitory concentration 50% (IC50) on the Y axis. Data show the gp120 Envs are good inducers of tier 1 HIV neutralizing antibodies.

FIG. 21 shows that immunization of guinea pigs with the immunogens on the X axis induces antibodies in plasma that bind to the Transmitted/founder Env 1086C, with binding levels on the Y axis shown in log Area under the curve (AUC) in ELISA binding assays.

FIG. 22 shows that immunization of guinea pigs with the immunogens on the X axis induces antibodies in plasma that bind to the gp120 Env C.TV1, with binding levels on the Y axis shown in log Area under the curve (AUC) in ELISA binding assays.

FIG. 23 shows that immunization of guinea pigs with the immunogens on the X axis induces antibodies in plasma that bind to the Env CAP174, with binding levels on the Y axis shown in log Area under the curve (AUC) in ELISA binding assays.

FIG. 24 shows that immunization of guinea pigs with the immunogens on the X axis induces antibodies in plasma that bind to the CAP174 V2 region peptide, with binding levels on the Y axis shown in log Area under the curve (AUC) in ELISA binding assays.

FIG. 25 shows that immunization of guinea pigs with the immunogens on the X axis induces antibodies in plasma that bind to the Env CAP260, with binding levels on the Y axis shown in log Area under the curve (AUC) in ELISA binding assays.

FIG. 26 shows that immunization of guinea pigs with the immunogens on the X axis induces antibodies in plasma that bind to the CAP260 V2 region peptide, with binding levels on the Y axis shown in log Area under the curve (AUC) in ELISA binding assays.

FIG. 27 shows that immunization of guinea pigs with the immunogens on the X axis induces antibodies in plasma that bind to the Ko224 Env gp120, with binding levels on the Y axis shown in log Area under the curve (AUC) in ELISA binding assays.

FIG. 28 shows that immunization of guinea pigs with the immunogens on the X axis induces antibodies in plasma that bind to the Ko224 V2 region peptide, with binding levels on the Y axis shown in log Area under the curve (AUC) in ELISA binding assays.

### DETAILED DESCRIPTION

In 2009, an HIV ALVACIAIDSVAX experimental vaccine Phase IIB efficacy trial in Thailand (RV144 trial) demonstrated an estimated 31.2% vaccine efficacy (Rerks-Ngarm et al, NEJM 361: 2209-2220 (2009). A recent immune correlates analysis of potential protective antibody responses in the trial demonstrated an inverse correlation of HIV-1 envelope VI V2 plasma antibodies with decreased infection risk (Haynes B F, Case Control study of the RV144 trial for immune correlates: the analysis and way forward. AIDS Vaccine 2011 (Bangkok, Thailand, 2011), Haynes B F et al, N. Eng. J. Med. April 2012, 366: 1275-1286). Thus, devising adjuvant and envelope formulations that generate higher levels of Env antibodies than those seen in RV 144 is a key goal of HIV vaccine development.

In the context of the Pox Protein Public Private Partnership (P5) in development is a proof-of-concept trial in South Africa, building on the findings of the RV144 trial but using clade C inserts and protein (gp120). The aim is to develop new envelope proteins and reagents for the subtype C HIV-1 epidemic. A pox virus (ALVAC) prime developed by Sanofi-Pasteur will contain the highly immunogenic ZM96gp120-TM (See Thongcharoen P, Suriyanon V, Paris R M, Khamboonruang C, de Souza M S, Ratto-Kim S, Karnasuta C, Polonis V R, Baglyos L, Habib R E, et al. J Acquir Immune Defic Syndr 2007, 46:48-55) and the boost will contain two subtype C gp120 proteins (TV-1 and 1086 C) formulated with the potent vaccine adjuvant MF59. Use of gp120 monomers instead of trimers has been prioritized in order to make comparisons with data from the RV144 trial (See Rerks-Ngarm S, Pitisuttithum P, Nitayaphan S, Kaewkungwal J, Chiu J, Paris R, Premsri N, Namwat C, de Souza M, Adams E, et al. N Engl J Med 2009, 361:2209-2220).

A bivalent SubtypeB/E Env gave ˜31% estimated vaccine efficacy in Thailand, a location where there was a recent founder virus effect resulting in remarkable heterogeneity of challenge AE viruses in the region. In contrast in South Africa, the SubtypeC have been around longer and are more diverse and the population will likely be of higher risk. A bivalent vaccine may not induce the needed breadth for the region to have a chance to show an effect.

In certain aspects, the invention provides additional envelope sequences, formulated as peptides, recombinant protein and nucleic acids, for use in the S. Africa efficacy trials. In certain embodiments, these sequences are used as a boost in the S. Africa efficacy trials. The rationale in choosing these envelope sequences was to identify sequences, Envs and/or peptides derived therefrom, preferably from acute subjects, thus approximating transmitted/founders (TFs)), wherein such sequences could complement the sequence coverage represented by the ZM651, TV1, and 1086c Envs that will be used for the S. African efficacy trial. These sequences are expected to complement the current sequence coverage in terms of providing complementary and more complete coverage of the V1V2 region. In certain aspects, the compositions of the invention enhance breadth in the upcoming S. African trial, wherein the V1V2 region is the likely target based on RV144 trial data. In certain aspects, the design of the compositions described herein also has the potential to enhance potency and the fraction of subjects that respond to the region.

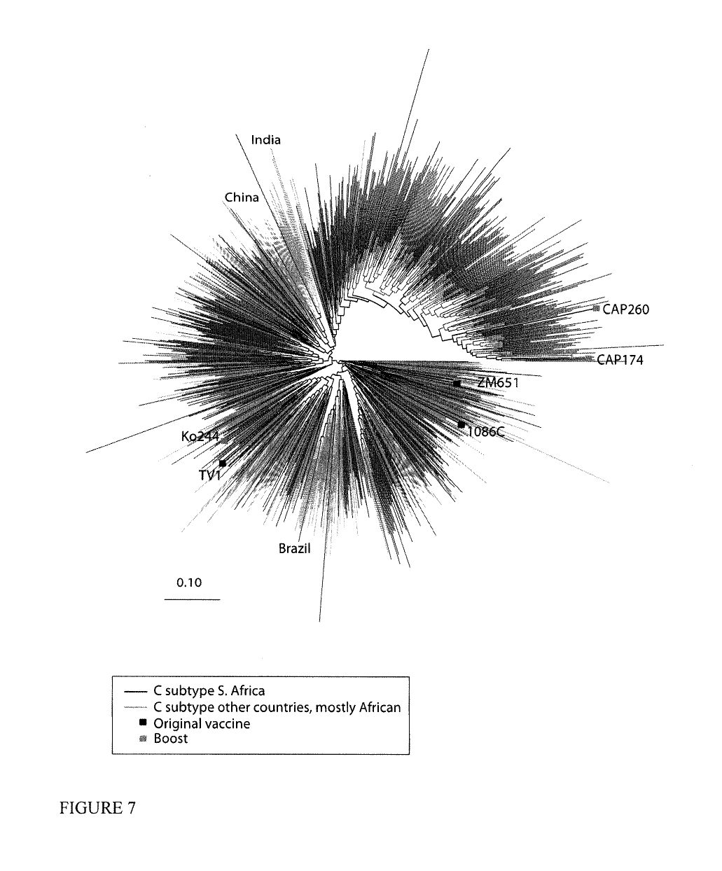

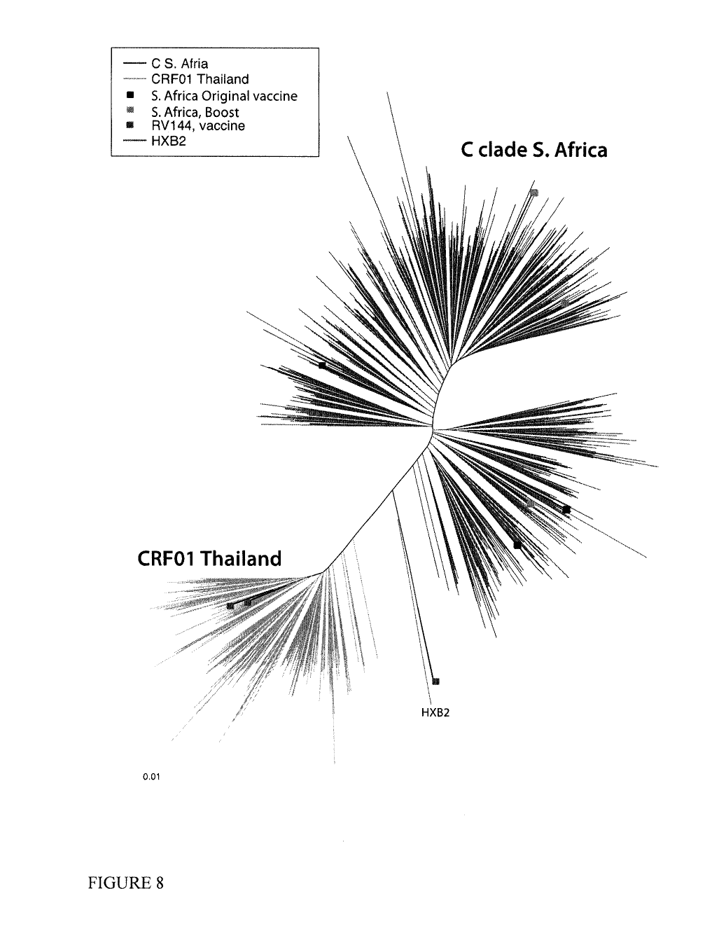

Enhancing the breadth of immunogens in the South African trial is desirable from several perspectives. First, the RV144 Thai trial was conducted in a significantly less diverse population than the S. African population (see phylogenetic analysis FIGS. 7-8). The most recent common ancestor of the Thai CRF01 epidemic was around 1988, while the C clade in S. Africa was more late 60s, and thus viruses have had an extra 20 years of diversification, and so the target population is more diverse. This impact is seen in serological panels—within-clade advantage comparing large panels of natural infection sera with large panels of isolates is much more profound for CRF01 and CRF07/CRF08, which is another epidemic with a more recent common ancestor, in China, than for B and C clades, more diverse older clades, where the within-clade advantage is fading. Thus his natural infection cross-neutralization studies indicates the challenge of the population diversity is greater in S. Africa than Thailand. This is despite the Thai epidemic being a 2 clade epidemic, as the CRF01 lineage so dominates.

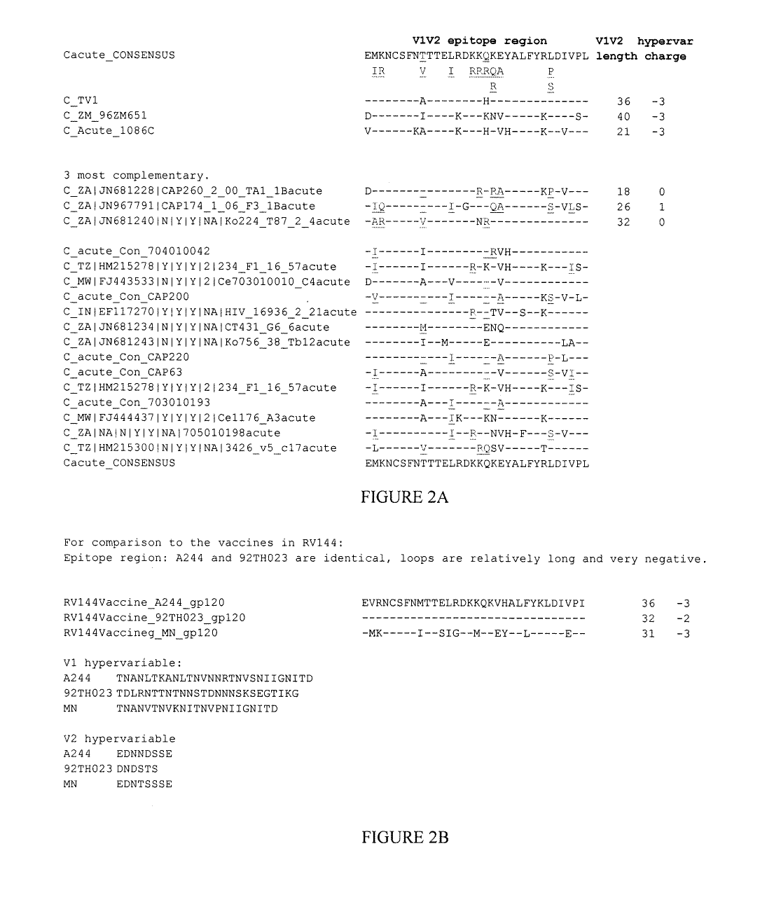

In the RV144 trial, the two CRF01 vaccine sequences were essentially identical in the stretch between the hypervariable loops where the antibodies bind (HXB2 153-184) (FIG. 2B), and though they differed somewhat from other regions of Env, they were both close to the MRCA (most recent common ancestor) of the Thai epidemic, so central and close to current variants in Thailand. That simple scenario cannot be mimicked in S. Africa, and if a vaccine response is elicited it will be up against a more variable viral population. Thus there is a need for better coverage of this prime vaccine target region. Without broader sequence coverage the beneficial effect in the Thailand trial might be harder to replicate in S. Africa population without giving the South African vaccine some advantages going in. The sequences as described herein, for example but not limited to their use as a boost, could provide better coverage of this prime vaccine target region.

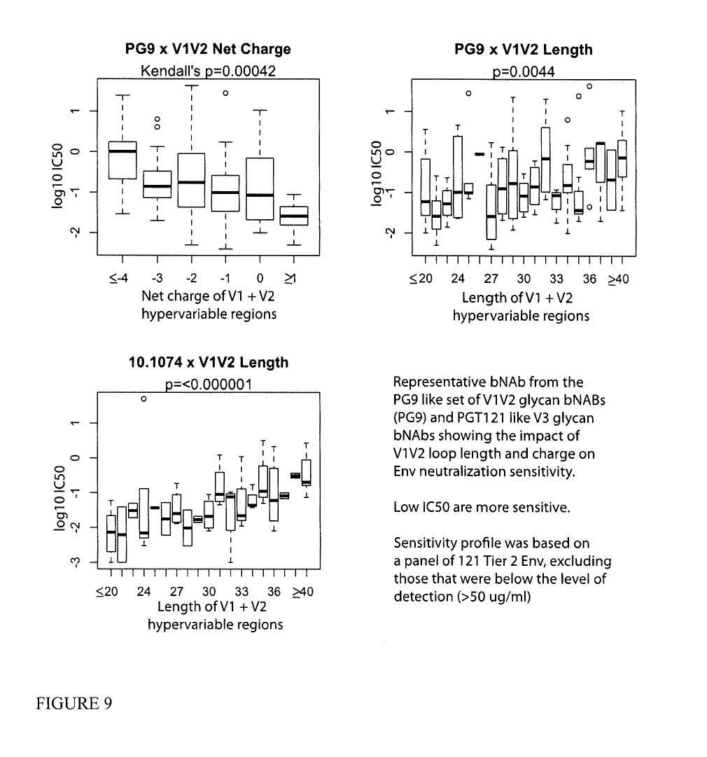

There are data to suggest that longer and more negatively charged V1-V2 hypervariable regions are associated with relative resistance to the PG9 class of antibodies. These hypervariable stretches surround the region of interest for PG9-like antibodies, and are not contact residues. Without being bound by theory, it is likely that the reason for this effect is that the longer loops generally restrict antibody access, and it is plausible that the negative charge tends to repel rather than draw them in. The PG-9 like antibodies do interact with the highly positively charged regions in the V2 region, and data show that they are all carry negative charge at the interface. The long V1V2 hypervariable stretches are also strongly associated with resistance of PGT121 like antibodies and have a mild negative impact on CD4 binding site antibodies. FIG. 9 is an example, how length and negative charge both affect binding. PG9-like antibodies to the V1V2 hypervariable region—for example HCDR3 length less than twenty four amino acids is unfavorable for PGT-like antibodies. FIG. 9 shows one example of each pattern for the class, but the patterns were quite consistent across antibodies in each class.

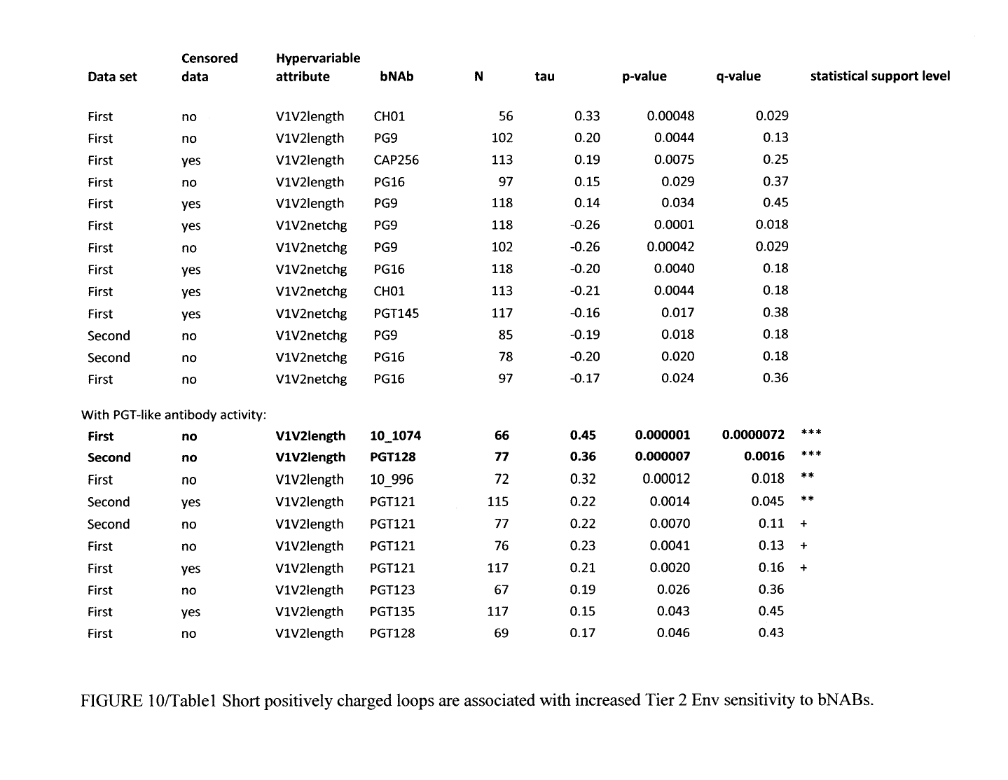

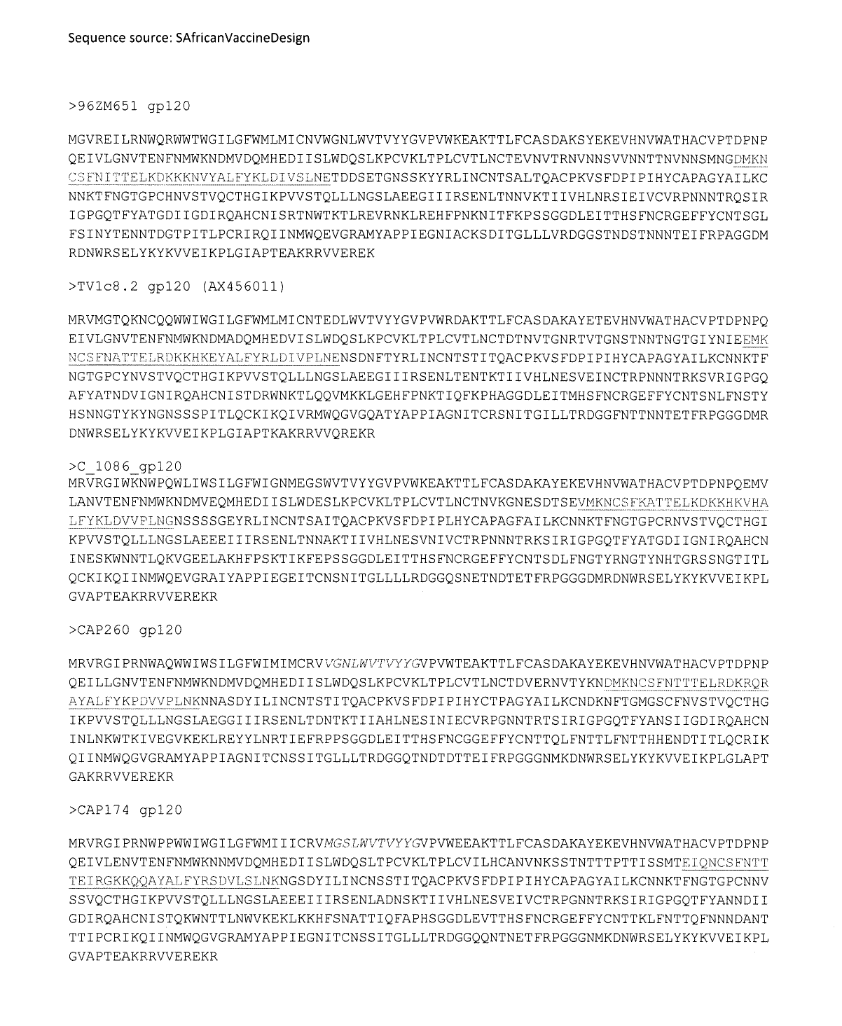

Based on the data presented in FIG. 9 and Table 1 (FIG. 10), the V1V2 region for vaccine candidates was divided into the 3 sections for analysis: V1 hypervariable, V2 hypervariable, and the region between, which is where PG9 like antibodies bind, as well as being the interesting target region in RV144.

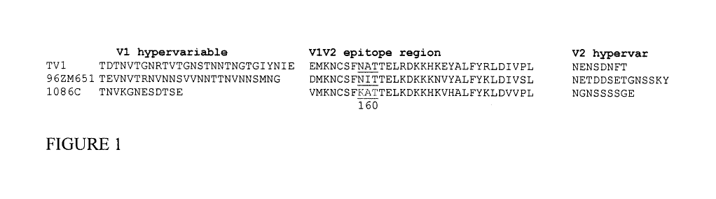

The vaccine current versions of these regions are shown in FIG. 1; the sequence of all three regions are contiguous, while in the figure the regions of interest are delineated by tabs.

FIG. 1 shows the V1V2 regions of the current vaccine envelopes. All three current vaccine component have undesirable net negative charge, and only 1086C has short hypervariable regions. Also, the glycosylation at position 160, critical for PG9 like neutralizing antibodies, is lost in the only sequence among the three sequences that also has short hypervariable regions.

In certain aspects, the invention provides additional reagents for use as immunogens in the South African vaccine. As discussed herein several parameters were considered in choosing additional envelope sequences. Envelope sequences were selected from acute clade C sequences, considered to be approximately representing transmitted founder virus sequence.

Described herein is one strategy for selection of additional envelope sequence to augment the South African vaccine design. Considerable data indicates that the V1V2 hypervariable loops strongly impact exposure of bNAb epitope exposure for both PG9 and PGT like bNAbs. Analyses of the RV144 trial, showed that less than half of the RV144 vaccine recipients made V2 antibodies, perhaps that could be enhanced by better exposure of the region. The analysis revisited hypervariable loop characteristics bNAb susceptibility patterns to guide cutoff selection. V1V2 hypervariable stretches considered together rather than separately provided the strongest correlates of sensitivity/resistance, so this region in clade C viruses is the focus of this analysis. As described herein, a set of sequences was selected from a subset of acute isolates with a net charge of zero or above, and hypervariable loops equal or <32 amino acids with a preference for shorter combinations. In some embodiments, the set of sequence can be used as a boost in addition to the current vaccine components.

Given the hypervariable region constraints described above, and the preference for the use of transmitted/founder Envs, and natural Envs (instead of consensus or mosaics), provided herein are three viruses that would in combination provide the maximum coverage of common amino acid substitutions in the PG9-like MAb and RV144 target regions in between the hypervariable regions in V1V2. The idea is that exposure to epitope variants may select for antibodies with greater breadth during affinity maturation, and that exposure to a single form is likely to drive type-specific antibodies—a hypothesis supported by Liao et al, Nature 2013, 496: 465-476.

Once the boost Envs were selected, phylogenetic analysis confirmed that they were not extreme outliers, which would be possibly less helpful in terms of other epitopes outside the region of interest, and that they cover genetics that are missed by the original three sequences.

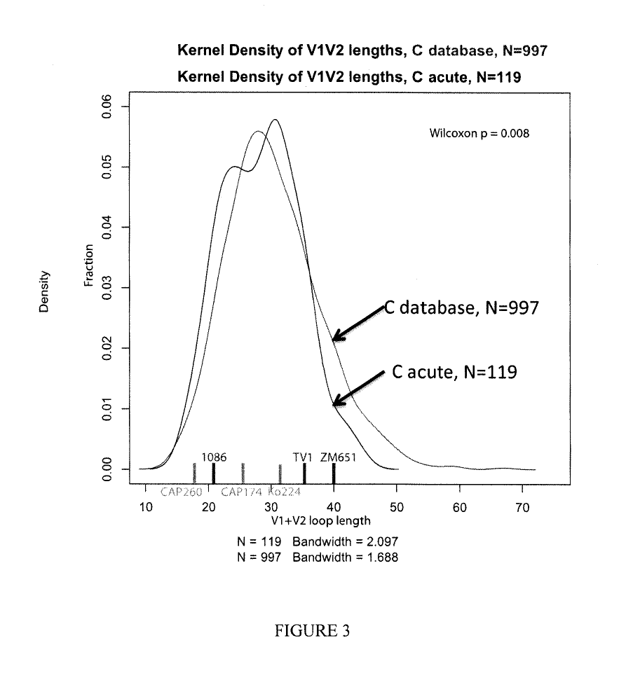

The length and charge of the V1V2 region were also considered. Regarding length, it is known that transmission of C subtype selects against very long loops—this pattern is maintained in the current data set. FIG. 3 shows the distribution of V1+V2 lengths in the HIV database of C subtype sequences, including only one sequence per person (some of whom are likely to be acute or early), versus those known to be derived from acute infection and TF or highly similar to TF viruses. The V1V2 length of the vaccine sequences are shown for comparison as nicks along the bottom, green (names below the nick) are the new suggestions, blue (names are above the nick) are the first three selected.

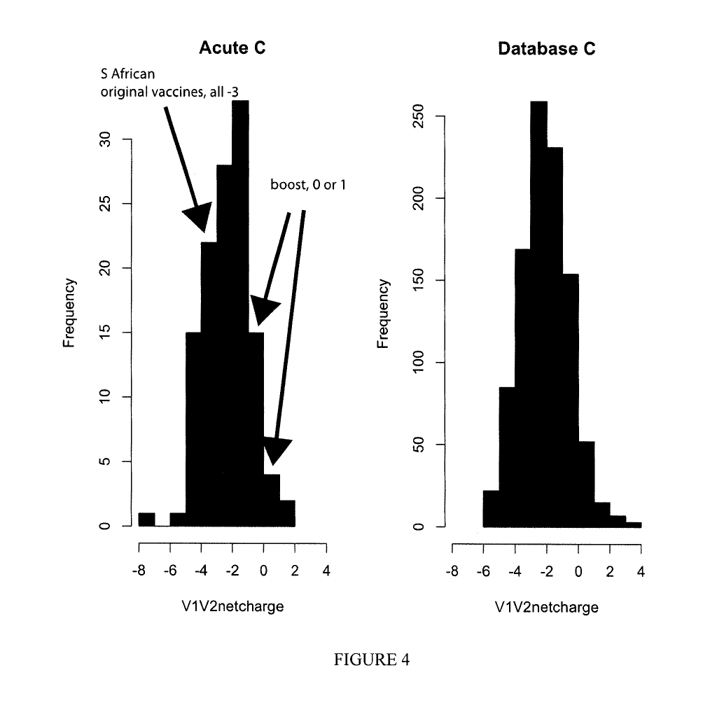

Regarding charge, FIG. 4 shows the net charge distribution among C clade acute sequences and the C clade database, there is a slight shift up at transmission, not significant, and the majority of sequences are negatively charged overall. As positively hypervariable loops are clearly important for PG9-like antibody sensitivity, a positive charge is desirable.

FIG. 2 shows sequences of a set of 17 C clade acute sequences from the V1V2 epitope contact region of interest, that met the criteria of having a V1V2 hypervariable region net charge that was higher or equal to zero, and a total length ≦32.

From these 17 sequences, three were selected that provide the best complementary coverage of the V2 region. These sequences provide excellent coverage of all of the most common amino acids in each position that are found in South Africa, thus coverage is not compromised by selecting Envs with relative short, positive V1V2 hypervariable regions.

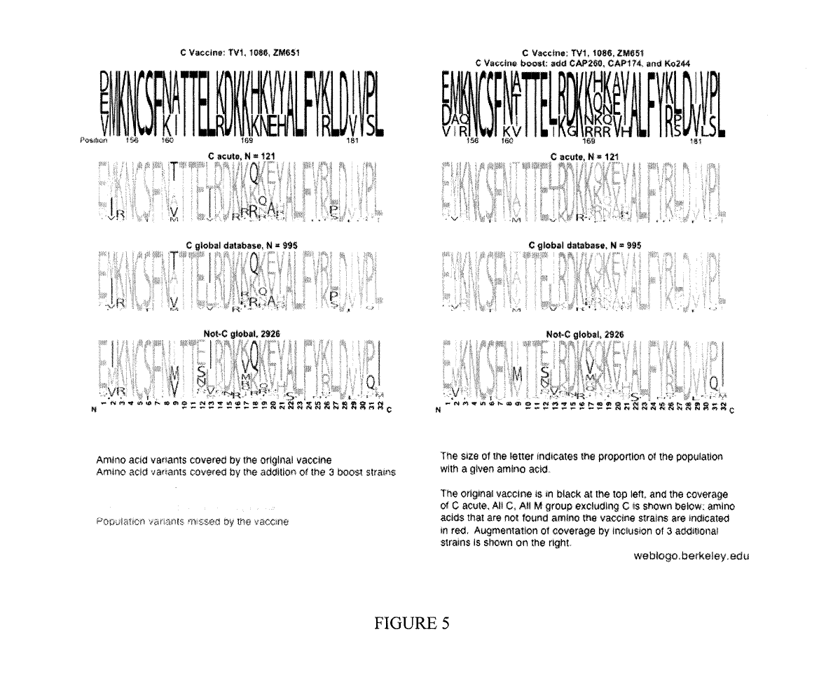

In FIG. 2, red/underlined amino acids in and under the consensus are common C clade variants that are missing from the initial (TV1, 96ZM651, 1086C) vaccine set. Red amino acids in the sequences themselves indicate amino acids not covered by the vaccine. Dashes are identity with the consensus. The coverage of different populations by different vaccine sets (three original versus three original+three in a boost) are also indicated in a LOGO plot (FIG. 5). The red amino acids on the left in the LOGOS are amino acids not captured in the original three vaccine set. The LOGO on the right shows what is gained in terms of vaccine coverage by adding three more sequences as described herein.

FIG. 2 also shows comparison to the sequences used in the vaccines in the RV144 trial. V1V2 epitope region: A244 and 92TH023 are identical, loops are relatively long and very negative. Despite being a correlate of protection, less than half the RV144 vaccinated people make V1V2 responses (See Haynes et al., NEJM (2012) 366: 1275-86). It is possible that long loops and negative charge in the V1V2 epitope region were limiting.

Phylogenetic analysis in FIG. 7 shows the location of the CAP 260, CAP174 and ko224 sequences compared to other clade C sequences.

The C clade envelope sequences (CAP260, CAP174, and ko224) are not extreme outliers of the C clade when introducing these three vaccines. Not only are they not outliers, but they really may have an unexpected benefit as part of the vaccine as well as enriching the V1V2 region, as 2 (CAP260 and CAP174) of the 3 fall into a very large S. African C clade sub-clade (FIG. 7) that was not well covered by the first 3 choices. The C clade phylogeny of FIG. 7 (maximum likelihood built with FastTree), shows where the vaccines are distributed relative to the C clade globally. About ½ of the S African sequences (in red below) form a distinctive clade. None of the three original vaccines are a member of that clade, and none are real outliers. The other ½ of the S. African sequences intermingle with other C clade sequences (non-S African C clade are orange), mostly from other regions of Africa, but a few from other countries with distinct lineages as indicated. None of our three original vaccines were part of that major S. African group (vaccine branches are blue boxes) while ⅔ of our boost strains are members of the large S. African C clade sub-lineage.

In certain aspects, the phylogenies also indicate these sequences would be excellent choices for an added boost for the S. African trial, given the constraints of using natural Envs and favoring transmitted/founders (FIG. 7). The phylogeny tree show that the three strains, and peptides as described, considered here to complement V1V2 are not extreme outliers, but they include representatives of a major sub-clade of S. African sequences that the original three vaccine strains missed.

FIG. 8 shows that the diversity challenge in South Africa is greater than was faced in Thailand, because the epidemic is older and has had longer to diverge. FIG. 8 shows a phylogenetic tree that compares S. African C clade isolates from the database (N=623 individuals) to Thai CRF01 isolates (N=245 individuals), with the vaccine strains indicated by colored boxes. The tree illustrates the differences in the divergence in the two populations.

Average amino acid distances (excluding gaps, so this is conservative) for:

CRF01 vaccines within clade in Thailand

### RV144Vaccine_A244× Thai CRF01=17.5%

### RV144Vaccine_92TH023=16.2%

B vaccine between clade comparisons, Thailand and S. Africa:

### RV144Vaccine_MN× Thai CRFO 1=31.1%

### RV144Vaccine_MN× S. Africa C=29.6

C clade vaccines within clade in S. Africa:

### TV1c8.2.× S. Africa C=22.8%

### 96ZM651× S. Africa C=22.1%

### 1086C× S. Africa C=22.1%

### CAP260× S. Africa C=22.0%

### CAP174× S. Africa C=20.6%

### Ko224_T87× S. Africa C=20.6%

The added sequences to the South African vaccine will bring in more common amino acids and potential epitopes in this setting.

The present invention includes the compositions disclosed herein and nucleic acids comprising nucleotide sequences encoding same. The proteins can be expressed, for example, in 293T cells, 293F cells or CHO cells (Liao et al, Virology 353:268-82 (2006), or any other suitable cell line. DNA, RNA, protein or vectored immunogens can be used alone or in combination. Methods to make synthetic peptides contemplated to be used in the compositions are also known in the art.

The compositions can be formulated with appropriate carriers using techniques to yield compositions suitable for administration. The compositions can include an adjuvant, such as, for example but not limited to, alum, poly IC, MF-59 or other squalene-based adjuvant, ASOIB or other liposomal based adjuvant suitable for protein or DNA immunization.

As indicated above, nucleic acid sequences (e.g., DNA sequences) encoding the immunogens can also be administered to a subject (e.g., a human) in formulations under conditions such that the immunogen is expressed in vivo and antibodies, including but not limited to BNAbs (broad neutralizing antibodies), are produced. The DNA can be present as an insert in a vector, suitable for recombinant protein expression, and/or for DNA vectors in a vaccination regimen. Non-limiting examples are a recombinant Adenoviral (Barouch, et al. Nature Med. 16: 319-23 (2010), recombinant mycobacterial (Le., BCG or M smegmatis) (Yu et al. Clinical Vaccine Immunol. 14: 886-093 (2007); ibid 13: 1204-11 (2006), or recombinant vaccinia type of vector (Santra S. Nature Med. 16: 324-8 (2010)). In non-limiting embodiments, the vaccinia vector is ALVAC, or NYVAC. Vector formulations can comprise any suitable adjuvant.

Immunogens of the invention, and nucleic acids (e.g., DNAs) encoding same, are suitable for use in generating an immune response (e.g., BNAbs) in a subject (e.g., a human patient) to HIV-1. The mode of administration of the immunogen, of encoding sequence, can vary with the particular immunogen, the patient and the effect sought, similarly, the dose administered. Typically, the administration route is intramuscular or subcutaneous injection (intravenous and intraperitoneal can also be used). Additionally, the formulations can be administered via the intranasal route, or intrarectally or vaginally as a suppository-like vehicle. Optimum dosing regimens can be readily determined by one skilled in the art. The immunogens (and nucleic acids encoding same) can be used prophylactically, however, their administration to infected individuals may reduce viral load. In certain embodiments, the compositions are administered to individuals who have been recently infected.

In accordance with the invention, immunization regimens can comprise administration of compositions as described herein (e.g., but not limited to an immunogen from FIG. 2 or Example 1) and can involve prime and boosts of combinations of such as peptides; gp120, gp140, gp160 immunogens, as a nucleic acid, in a suitable delivery vector, or a protein. In certain embodiments, the envelopes are recombinant. In certain embodiments, the envelopes are used as monomers.

Various assays are known in the art to test immunogenicity of the inventive compositions and regimens. Such assays include but are not limited to analyzing qualitative and quantitative response. For example but not limited to analyzing antibody titers and avidity, specificity, breadth of antibodies induced by immunization. Sera from immunized animals at various time points can be analyzed by ELISA for binding Abs, avidities; for MAb competition by ELISA to map epitope recognition; for Mab competition assay to map epitope recognition; for virus neutralization in TZM-bl and A3R5 assays.

### EXAMPLES

### Example 1

### Non-Limiting Examples of Envelope Sequences Contemplated by the Invention

V1V2 peptides for immunization. A set of 32-aa-long peptides with the full sequence from the peptides of FIG. 1 is made. In certain embodiment, the cysteine is changed to Serine, for example, so as not to dimerize. In certain embodiments, these peptides are biotinylated for ELISA assays.

C4-V1V2 peptides for immunization. A set of 22-aa-long V1V2 peptides is made, where a peptide is starting at LRDKK (or sequence in the corresponding position) and having two natural amino acids sequence beyond the “PL” (or sequence in the corresponding position) so as to get a potential Th epitope. These peptide comprise T-helper determinants from the fourth constant (C4) gp120 region. See Bartlett et al. AIDS (1998), 12: 1291-1300, at 1292. In certain embodiments, the C4 sequence is KQIINMWQEVGKAMYA (SEQ ID NO: 73). In certain embodiment, these peptides comprise N-terminal C4 sequence. In certain embodiment, these peptide comprise C-terminal C4 sequence. In certain embodiment, these peptides are expected to produce at least 75% response rate to the peptide.

CAP260 peptides:
(SEQ ID NO: 61)
DMKNCSFNTTTELRDKRQRAYALFYKPDVVPLNK
(SEQ ID NO: 62)
DMKNCSFNTTTELRDKRQRAYALFYKPDVVPL
(SEQ ID NO: 63)
DMKNSSFNTTTELRDKRQRAYALFYKPDVVPL
(SEQ ID NO: 64)
C4-LRDKRQRAYALFYKPDVVPLNK
CAP174 peptides:
(SEQ ID NO: 65)
EIQNCSFNTTTEIRGKKQQAYALFYRSDVLSLNK
(SEQ ID NO: 66)
EIQNCSFNTTTEIRGKKQQAYALFYRSDVLSL
(SEQ ID NO: 67)
EIQNSSFNTTTEIRGKKQQAYALFYRSDVLSL
(SEQ ID NO: 68)
C4-IRGKKQQAYALFYRSDVLSLNK
Ko224 peptides:
(SEQ ID NO: 69)
EARNCSFNVTTELRDKNRKEYALFYRLDIVPLNE
(SEQ ID NO: 70)
EARNCSFNVTTELRDKNRKEYALFYRLDIVPL
(SEQ ID NO: 71)
EARNSSFNVTTELRDKNRKEYALFYRLDIVPL
(SEQ ID NO: 72)
C4-LRDKNRKEYALFYRLDIVPLNE

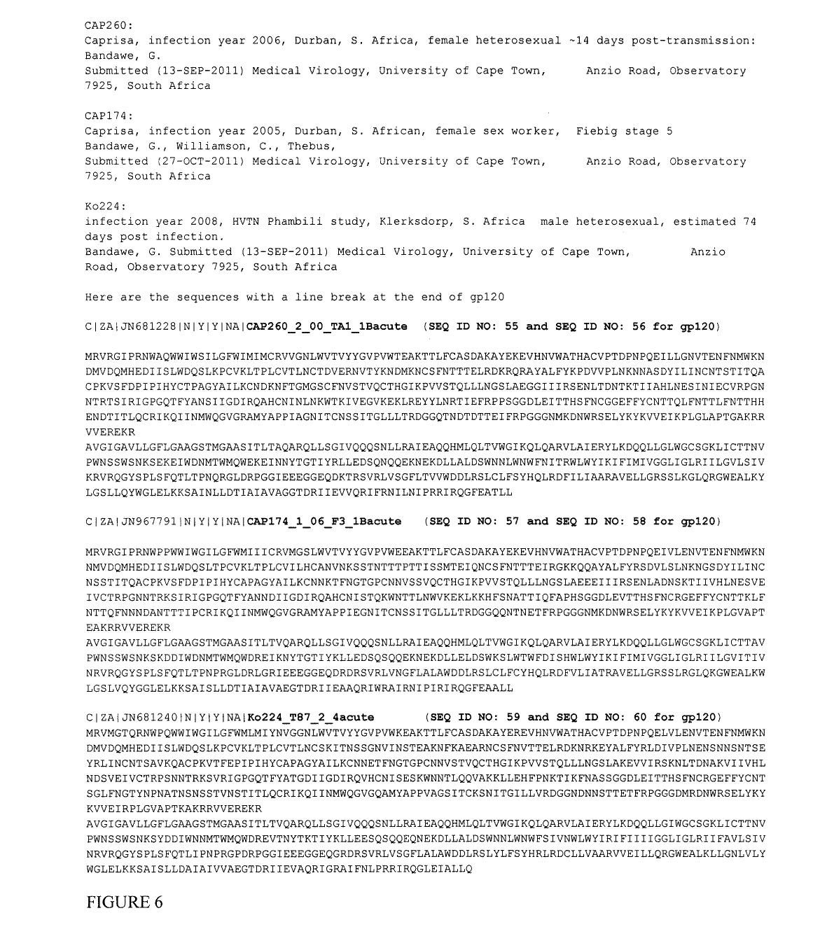

Protein envelope sequences, as a full length or gp120, are presented in FIG. 6.

### Example 2

### Immunization Studies

A model of the immunization strategy will be tested in non-human animals, e.g. mice, guinea pigs, and/or macaques, to compare the use of V1V2 peptides and/or envelopes, for example gp120s as described herein, for example as a boost, in conjunction with the original vaccine design, using the originally proposed formulations (e.g. using MF59 as an adjuvant) and delivery methods.

Some consideration include the choice of peptides or envelopes as immunogens to augment the SA ALVAC-ZM651/TV-1/1086C C/C boost. A comparison study in Non-human primates (NHPs) of peptides vs. Envs can address this question. The key issues to peptide immunogenicity are size of the peptides, the need for additional “universal” T helper epitopes and peptide immunogenicity in the adjuvant to be used. We have performed two human clinical trials with a HIV Th epitope from the C4 Env region conjugated N-terminal to a V3 peptide. In infected individuals 62.5% of subjects responded with T cell responses and 50% with neutralizing antibody increases (See Bartlett et al. AIDS (1998), 12: 1291-1300). In uninfected subjects, the C4-V3 peptide induced tier 1 neutralizing antibody responses in 75% of vaccinees, and CD8+CTL in 29% of vaccinees. This peptide vaccine in Seppic Montanice ISA-51 oil adjuvant was safe and non-reactogenic in HIV infected subjects (0.25 ml dose deep IM per site) but caused sterile abscesses in seronegative subjects (0.5 ml sub cut. per site). Thus, peptides can be very immunogenic in the right adjuvant. The inventive peptide will be tested in the adjuvant to be used in the South African trial.

The inventive peptides contain V2, and now there is have a report of V3 antibodies correlating with decreased transmission risk in RV144 (PLoS One 8: e75665, 2013). In RV144 clinical trial Env immunization was quite immunogenic and Env IgG to V1V2 was an immune correlate. There are observation that there is a V3 site of immune pressure in the global sieve analysis of RV144. Envs induced the putative protective antibodies and Env should be considered for immunization and for comparison to peptides immunogenicity in NHPs.

Choice of adjuvant. Choices of adjuvants include AS01B, MF-59 and IDRI GLA/SE and GLA/Liposomes/QS21 (similar to ASO1B from IDRI).

Vaccine strategies include but are not limited to the following rhesus macaques studies:

Group 1:

Original design—ZM96-gp120 ALVAC prime, followed by boost with gp120 (TV-1 and 1086C) formulated with the MF-59 adjuvant. Trivalent Envs clade C (CAP260, CAP174, Ko224) in adjuvant is administered IM. Three dose groups. 30 ug each Env, 100 ug each Env, 300 ug each Env; each dose group 4 animals.

Group 2:

Original Design+boost with the combination of three peptides from CAP260, CAP174, Ko224 (Cysteine to Serine modified) formulated with a suitable adjuvant, for example the MF-59 adjuvant. Trivalent V2 peptides in adjuvant administered IM. Three dose groups 30 ug each peptide, 100 ug each peptide, 300 ug each peptide; each dose group 4 animals.

Group 3:

Original design+boost with the combination of three peptides from CAP260, CAP174, Ko224 (C4-V2 peptides) formulated with a suitable adjuvant, for example the MF-59 adjuvant;

Group 4:

Original design+boost with combination of three gp120s CAP260, CAP174, Ko224 (SEQ ID NOs: 56, 58, 60) formulated with a suitable adjuvant, for example the MF-59 adjuvant. Trivalent C4-V2 peptides in adjuvant administered IM. Three dose groups. 30 ug each peptide, 100 ug each peptide, 300 ug each peptide; each dose group 4 animals.

The CAP260, CAP174, Ko224 peptide and envelope sequences can be used in multiple boosts. In certain embodiments, the regimen includes at least 3 boosts. A skilled artisan can readily determine the number and frequency of boosts. In a non-limiting embodiment, each group receives 4 immunizations, each 6 weeks apart.

In other embodiments, the regimen contemplates adding the CAP260, CAP174, Ko224 peptides and/or envelopes earlier in the immunization regimen, for example as a prime, alone or in addition to ZM96-gp120 prime of the original design.

In certain embodiments, the invention provides a pentavalent immunogen as an arm of the South African trial. Certain embodiments provide three peptides and/or envelopes to be added to the Subtype C/C boost of TV1 and 1086C envelopes. The global diversity of Subtype C in South Africa was analyzed and three additional Envs were selected, and peptides therefrom were designed, that will improve general Env diversity and will improve sequence coverage and diversity at V2.

In certain embodiments the invention provides trivalent peptide designs for adding to the Envs in the current design, as well as making additional gp120s immunogens to be added to the Envs in the current design. In certain embodiments, the Subtype C/C Env gp120s (TV1+1086C) is compared with Subtype C/C/C/C/C Env gp120 (TV1+1086C+the three SubtypeCs CAP260, CAP174, Ko224 env as gp120s or peptides) as boost in guinea pigs, or any other suitable animal model, to determine which regimen is more immunogenic. In other embodiments, comparison is made between regimens which use the gp120 Envs or the peptides therefrom as additions to the current design. For V2 there are some data with the Subtype AE Envs vs. short polypeptides of V1V2 that the Envs induce more neutralizing and ADCC antibodies than the peptides.

A NHP study can determine whether the C/C/C/C/C boost improves ADCC and/or neutralizing antibodies compared to C/C Env gp120s (TV1+1086C). All documents and other information sources cites herein are incorporated by reference in their entirety.

### Example 3

### Study of Role of V2 Peptides in Augmenting Antibody Responses Induce by the C.TV-1 and C.1086 gp120 Envs

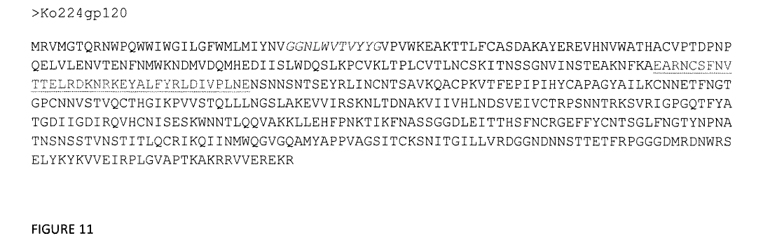

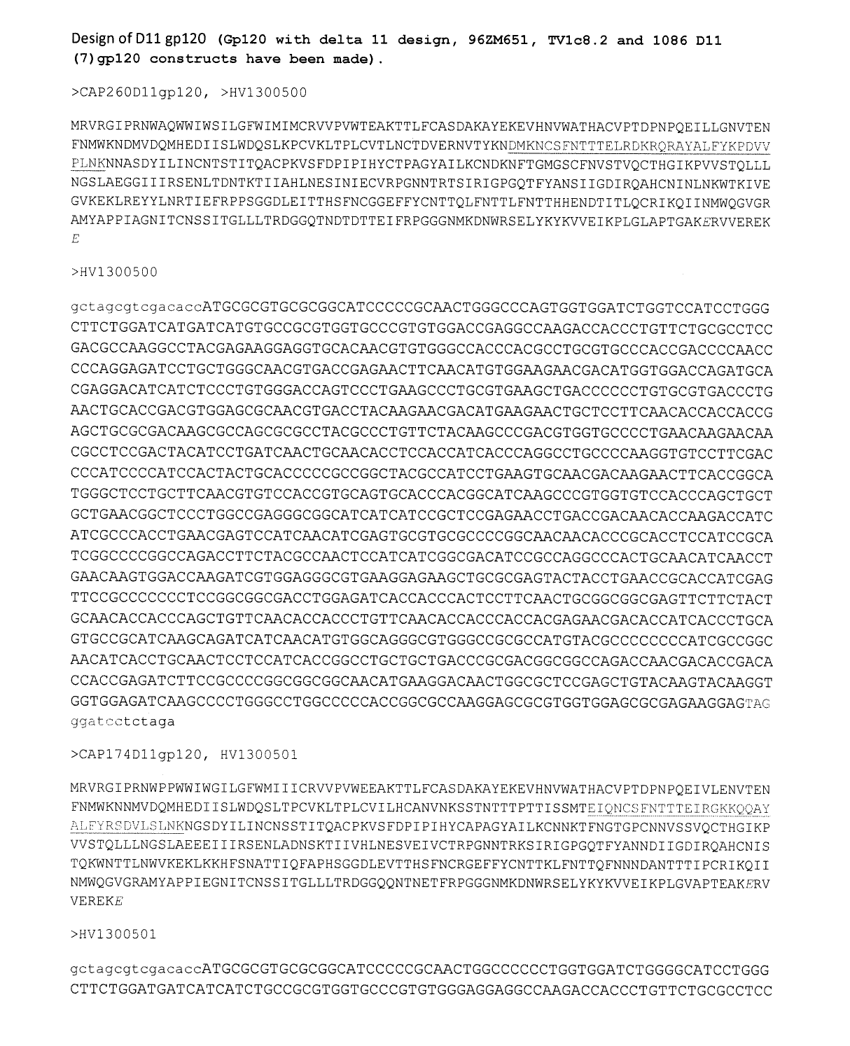

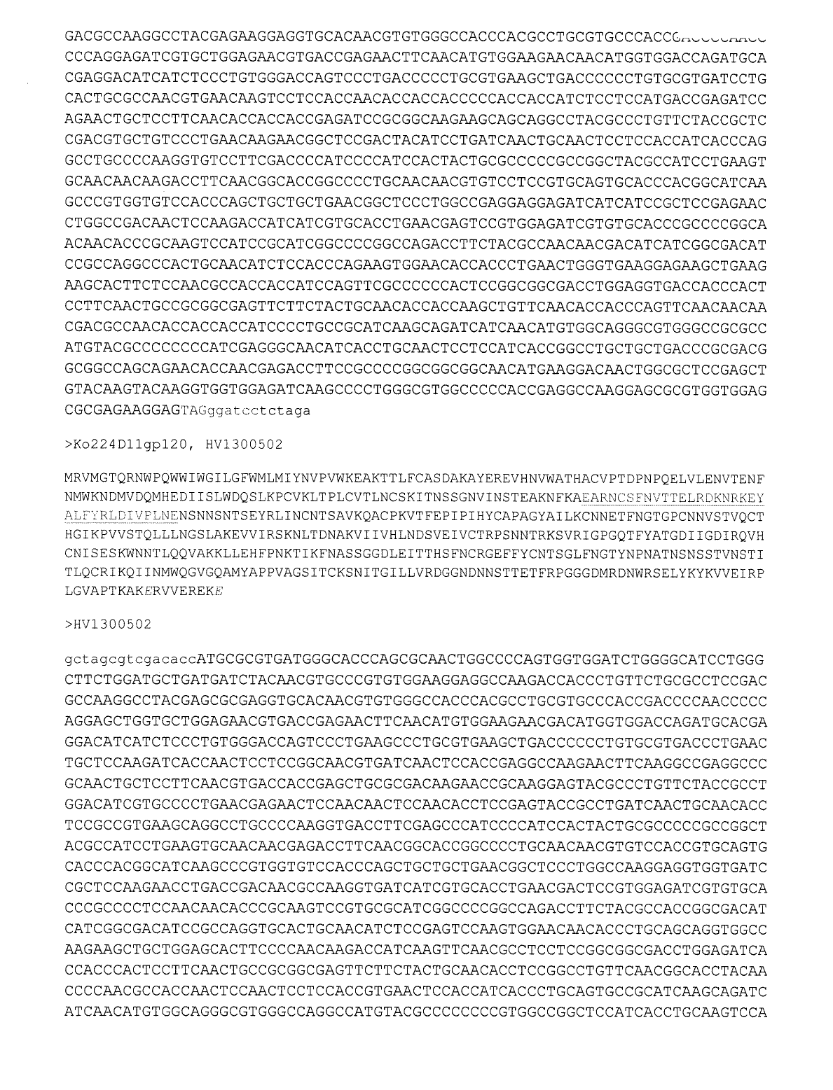

Sequences are shown inter alia in FIGS. 1, 2, 11, 12, and 13, and SEQ ID NOs: 61-72.

The following groups of animals were studied with the prime and boosts for each group those listed after each group name.

Group 1—(489) Trivalent V2 peptides, C.CAP260, C.CAP174, C.Ko224;

Group 2—(490) Trivalent C4-V2 peptides, C.CAP260, C.CAP174, C.Ko224;

Group 3 (491) Trivalent gp120 Envs, C.CAP260, C.CAP174, C.Ko224;

Group 4 (492) C.TV1+C.1086C gp120 Envs;

Group 5 (494) Pentavalent gp120 Envs—C.TV1, C.1086C, C.CAP260, C.CAP174, C.Ko224 gp120s;

Group 6 (495) C.TV1+C.1086C gp120 Envs plus Trivalent V2 peptides, C.CAP260, C.CAP174, C.Ko224.

Regimen: 100 ug total dose each immunization per guinea pig; if three immunogens, 34 ug per Immunogen: if 5 immunogens 20 ug per immunogen i.e. Total dose of Env/peptide kept the same per immunization.

Adjuvant: Stable emulsion with TLR9 (Type B oCpG, 10103) and TLR7/8 (R848).

Dosing intervals: three immunizations on Days 0, 21, and 42.

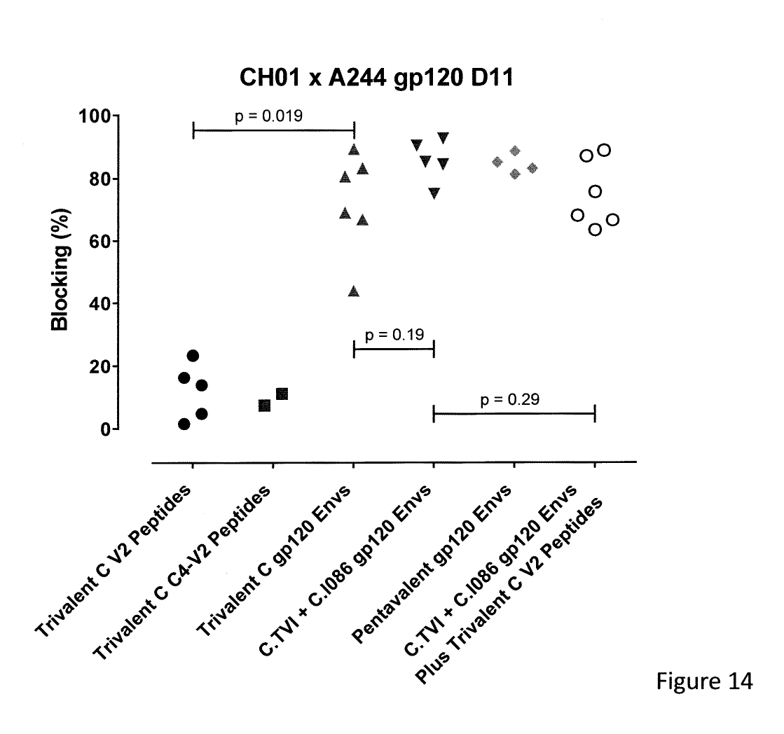

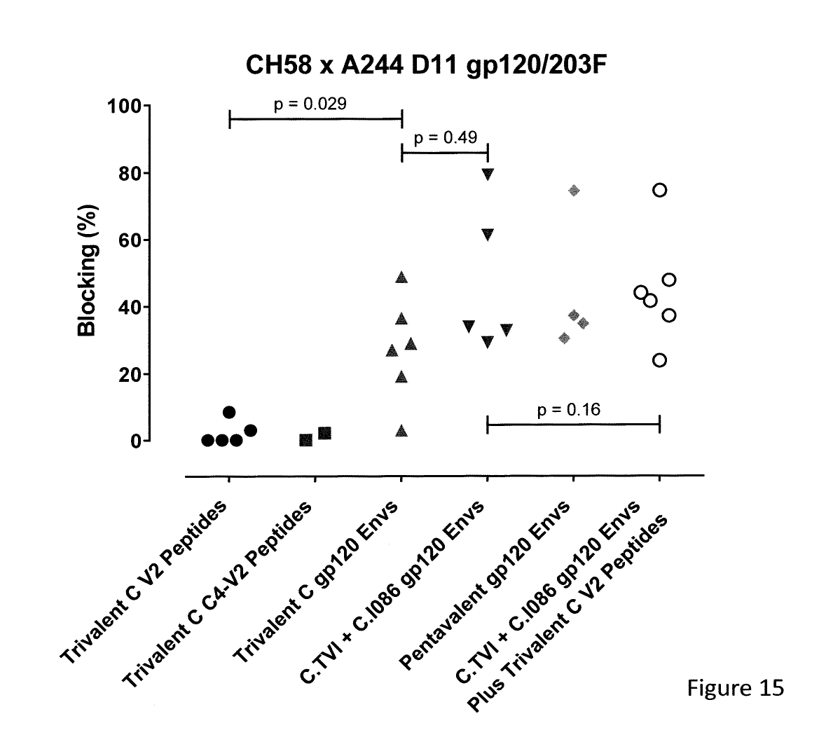

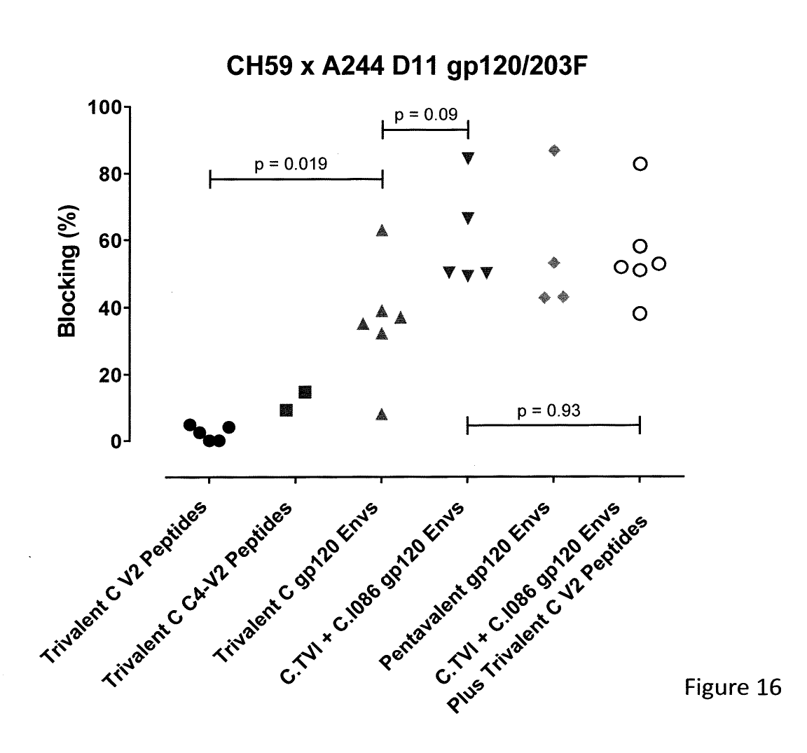

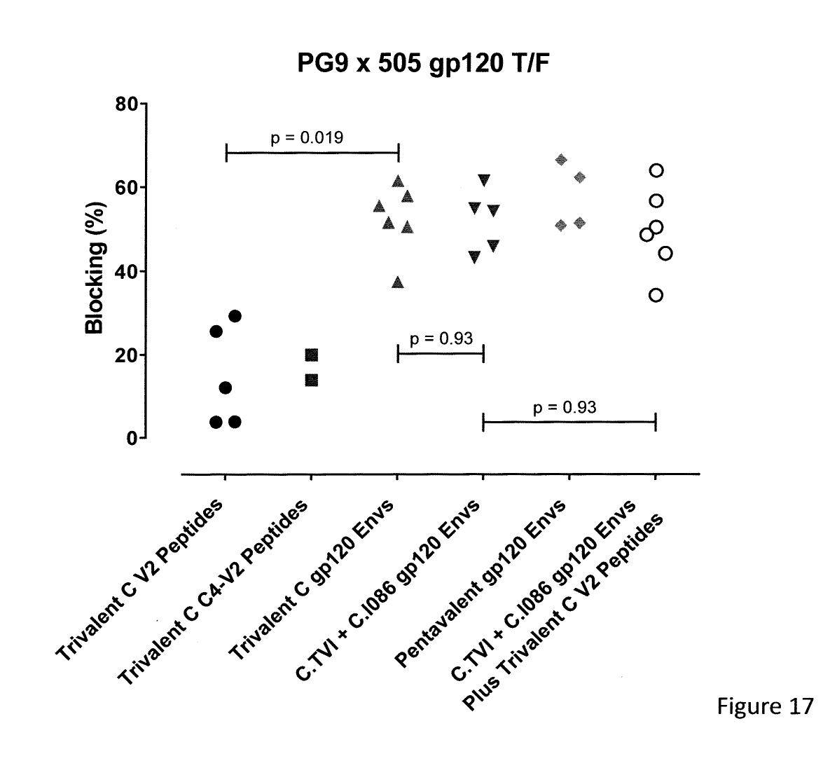

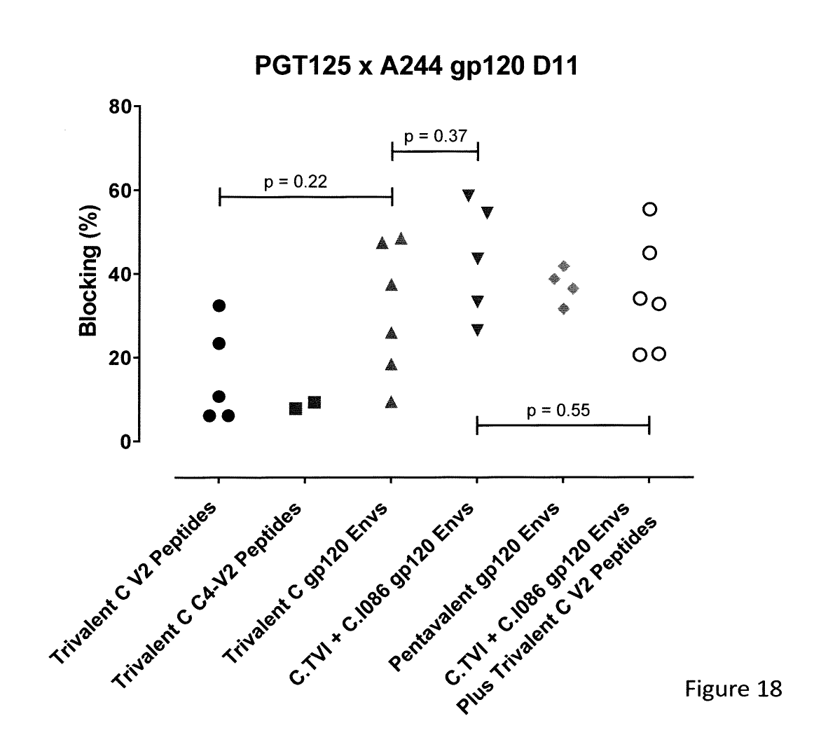

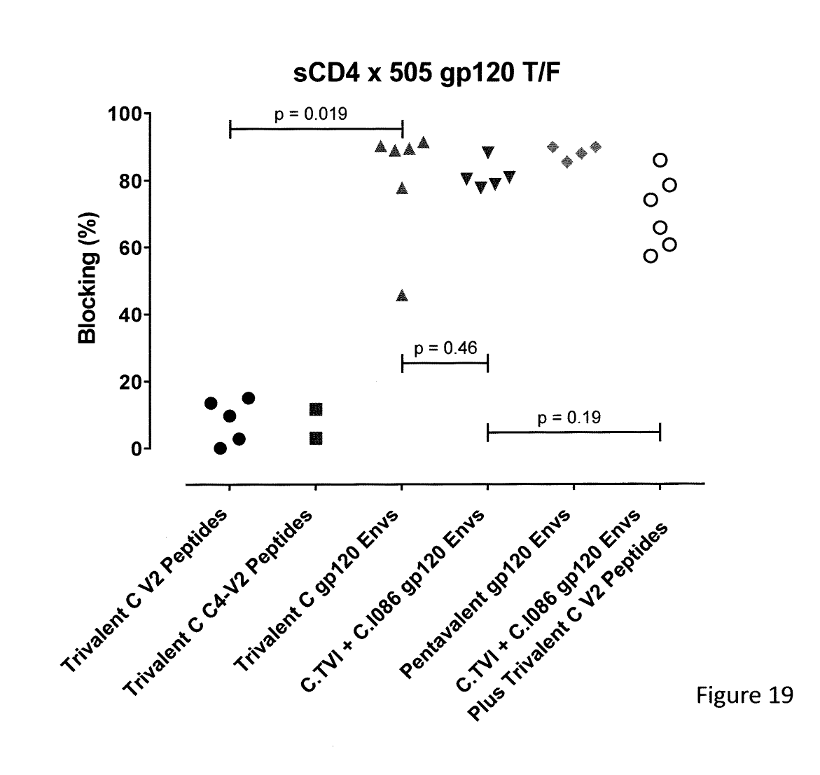

Blocking Assays of Immunized Guinea Pig Plasma: (FIGS. 14-19)

Part 489: Trivalent V2 peptides, C.CAP260, C.CAP174, C.Ko224

Part 490: Trivalent C4-V2 peptides, C.CAP260, C.CAP174, C.Ko224

Part 491: Trivalent gp120 Envs, C.CAP260, C.CAP174, C.Ko224

Part 492: C.TV1+C.1086C gp120 Envs

Part 494: Pentavalent gp120 Envs—C.TV1, C.1086C, C.CAP260, C.CAP174, C.Ko224 gp120s

Part 495: C.TV1+C.1086C gp120 Envs plus Trivalent V2 peptides, C.CAP260, C.CAP174, C.Ko224

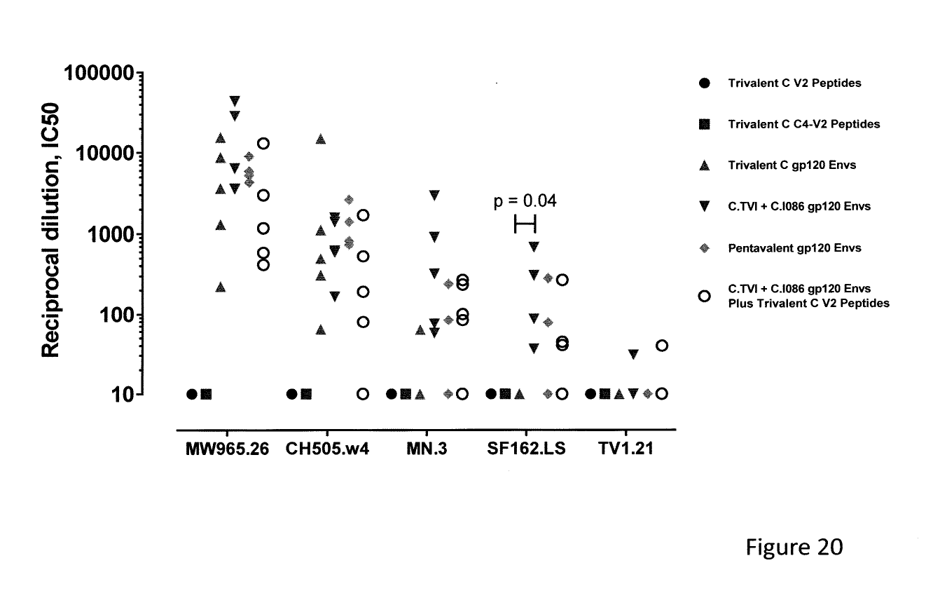

Neutralization Assays of Immunized Guinea Pig Plasma: (FIG. 20)

Part 489: Trivalent V2 peptides, C.CAP260, C.CAP174, C.Ko224

Part 490: Trivalent C4-V2 peptides, C.CAP260, C.CAP174, C.Ko224

Part 491: Trivalent gp120 Envs, C.CAP260, C.CAP174, C.Ko224

Part 492: C.TV1+C.1086C gp120 Envs

Part 494: Pentavalent gp120 Envs—C.TV1, C.1086C, C.CAP260, C.CAP174, C.Ko224 gp120s

Part 495: C.TV1+C.1086C gp120 Envs plus Trivalent V2 peptides, C.CAP260, C.CAP174, C.Ko224

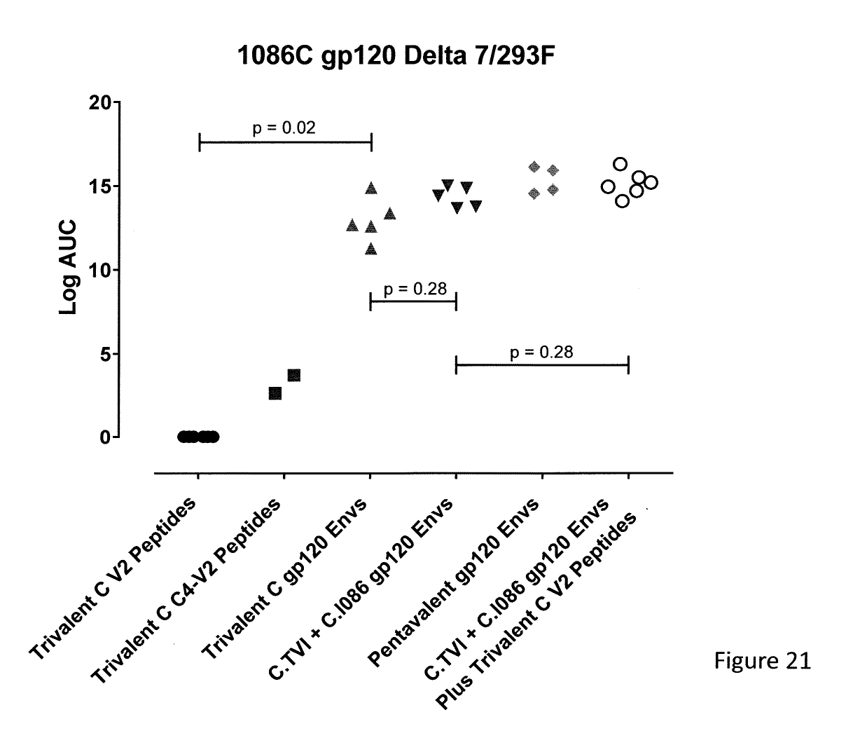

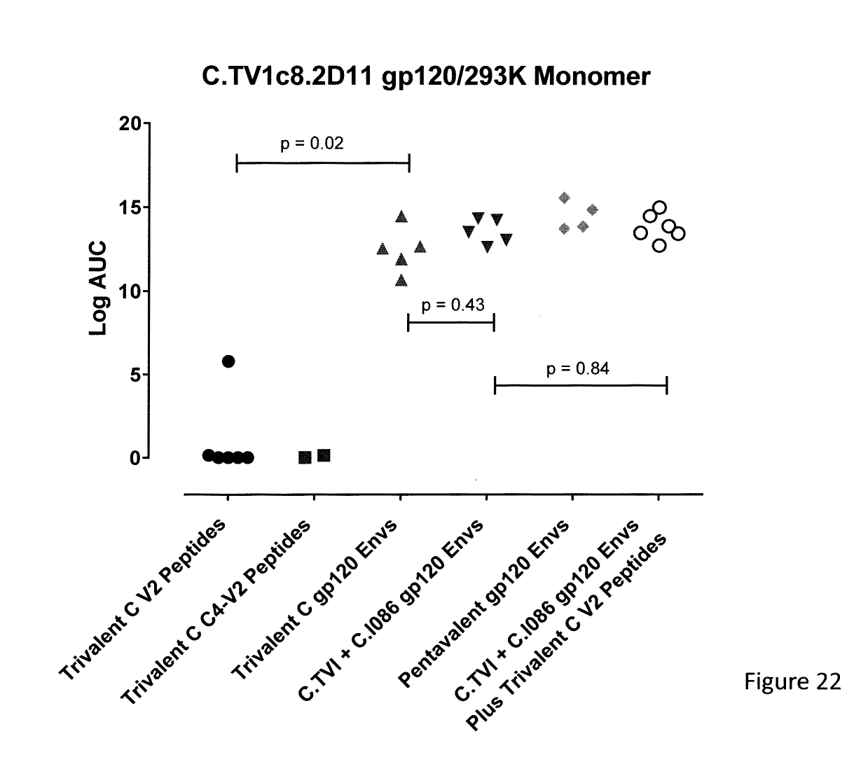

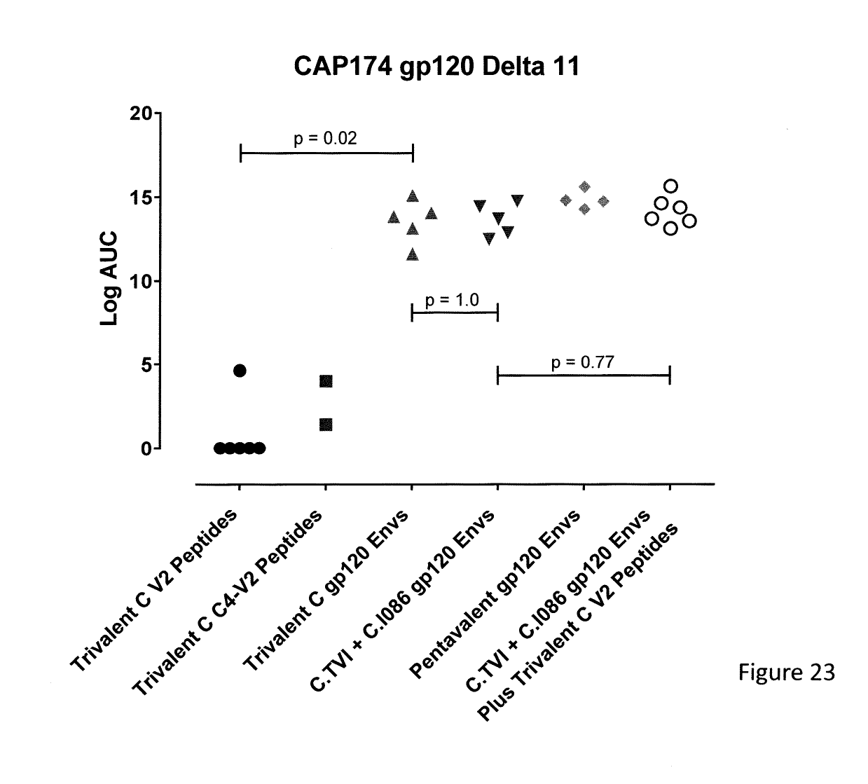

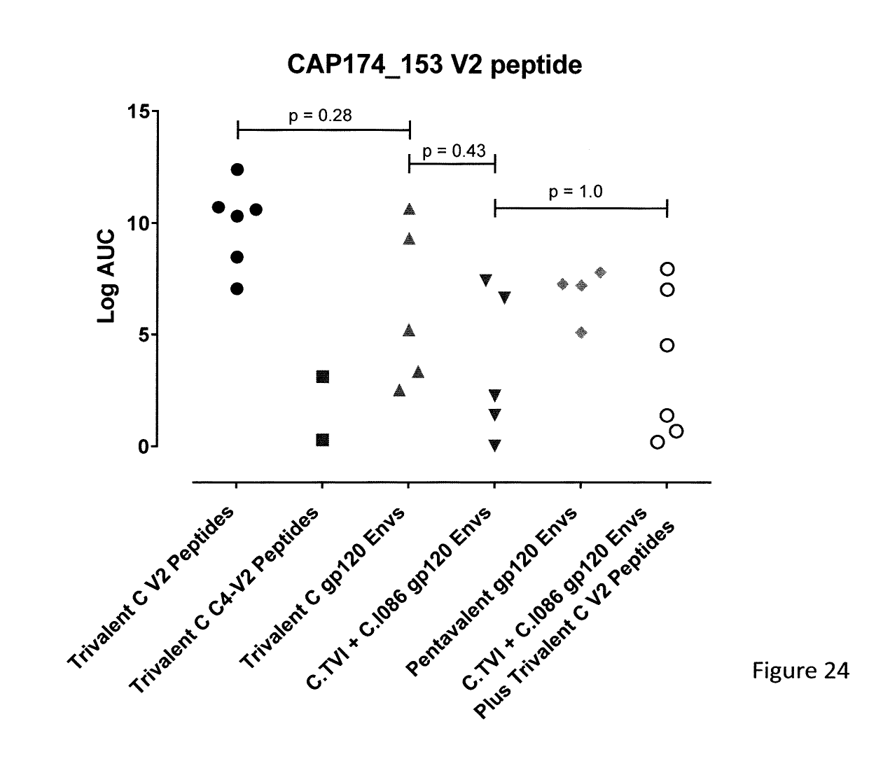

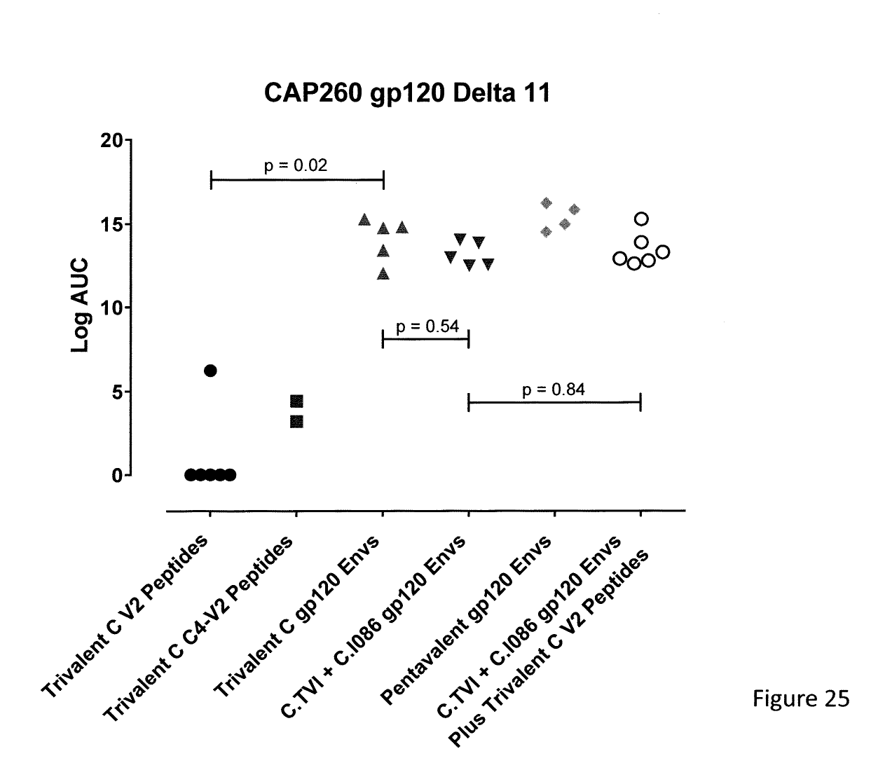

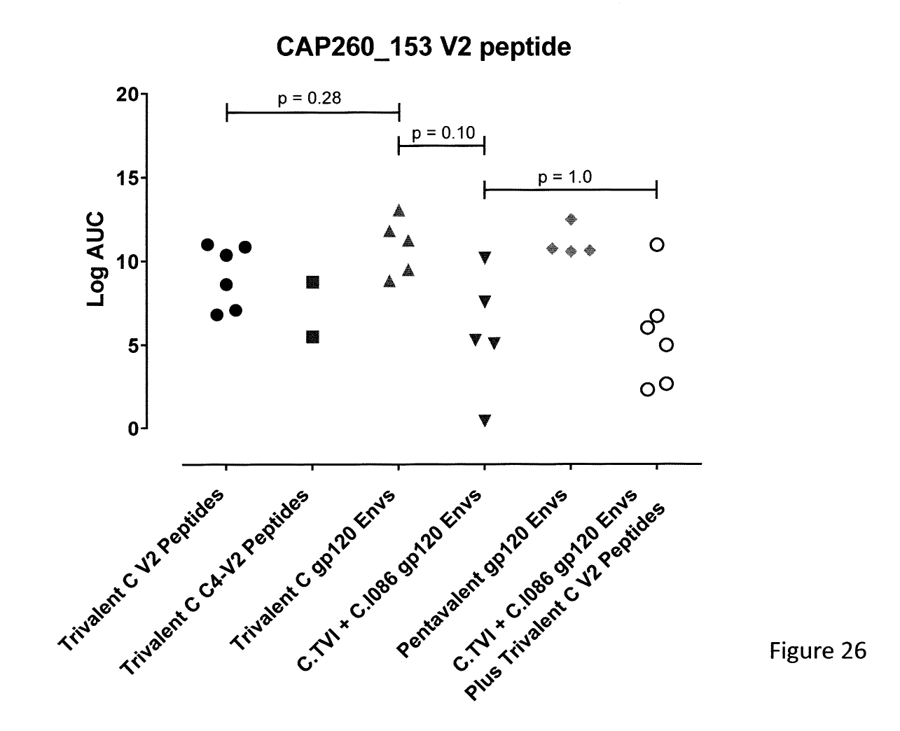

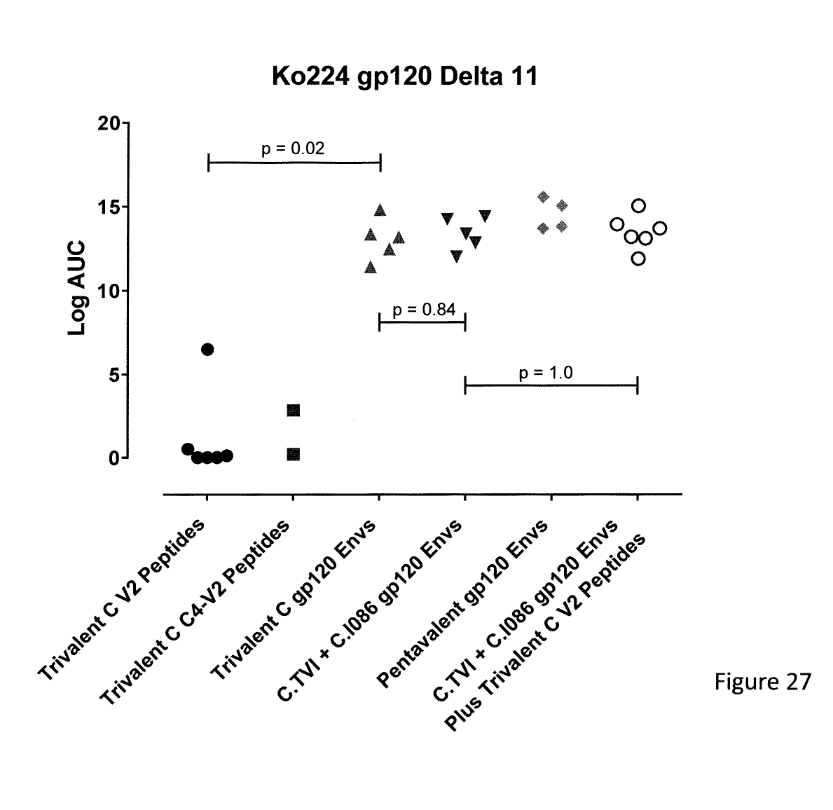

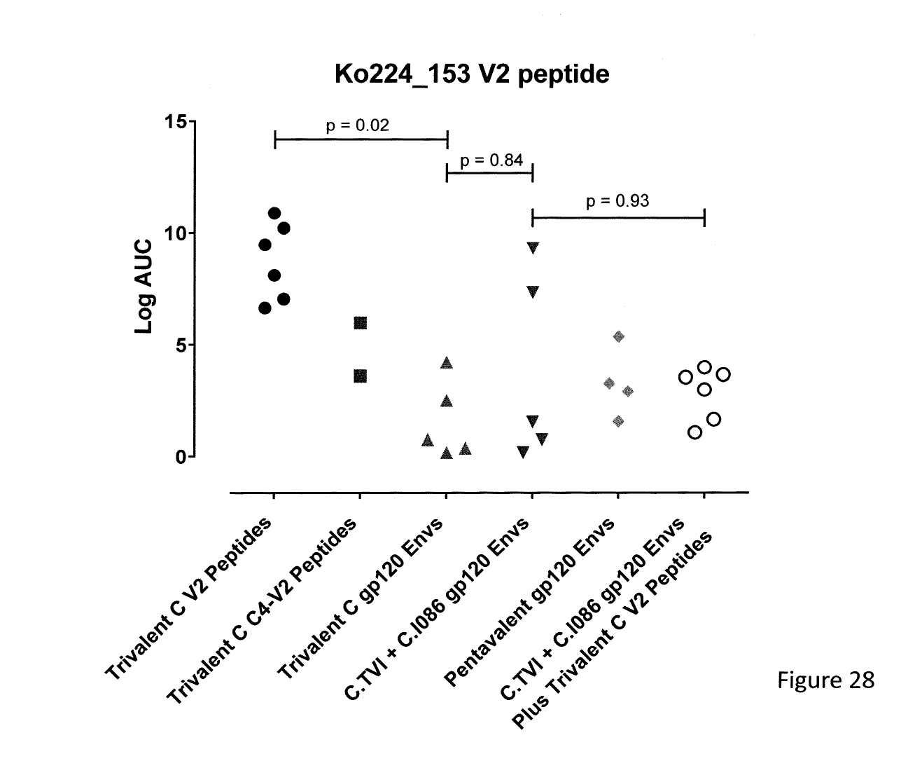

Binding Assays of Immunized Guinea Pig Plasma: (FIGS. 21-28)

Part 489: Trivalent V2 peptides, C.CAP260, C.CAP174, C.Ko224

Part 490: Trivalent C4-V2 peptides, C.CAP260, C.CAP174, C.Ko224

Part 491: Trivalent gp120 Envs, C.CAP260, C.CAP174, C.Ko224

Part 492: C.TV1+C.1086C gp120 Envs

Part 494: Pentavalent gp120 Envs—C.TV1, C.1086C, C.CAP260, C.CAP174, C.Ko224 gp120s

Part 495: C.TV1+C.1086C gp120 Envs plus Trivalent V2 peptides, C.CAP260,

C.CAP174, C.Ko224.

The data in Example 3 demonstrate that three new Envs were more immunogenic than the peptides.

### Example 4

Immunizations as described in Example 3 will be carried wherein the dose of each immunogen is 10 ug, i.e. the total dose would be 300 ug if three immunogens, 500 ug if five immunogens.

What is claimed is:

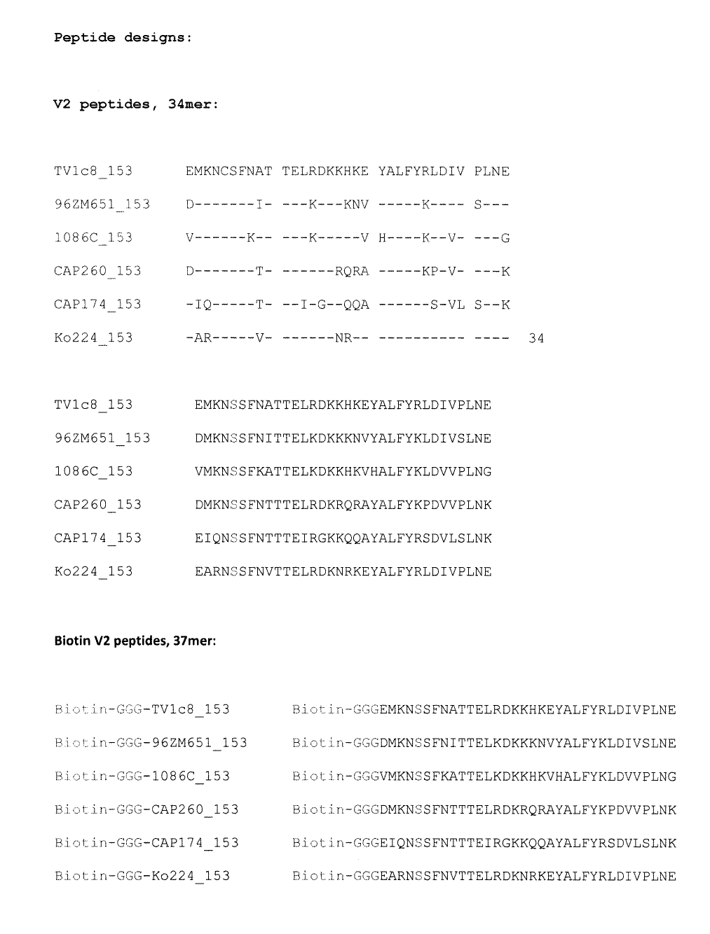

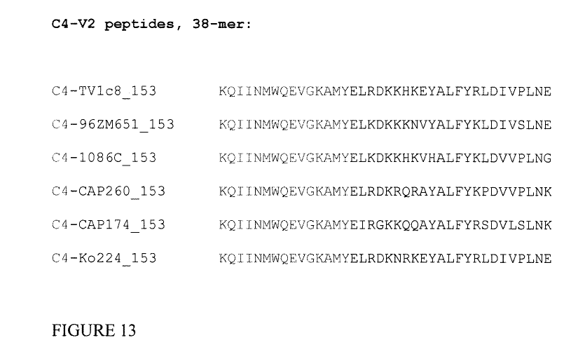

1. A synthetic peptide of FIG. 2 (SEQ ID NOs: 8-10, 29-31), or Example 1 (SEQ ID NO: 64, 68, 72), or FIG. 13, or a combination thereof.

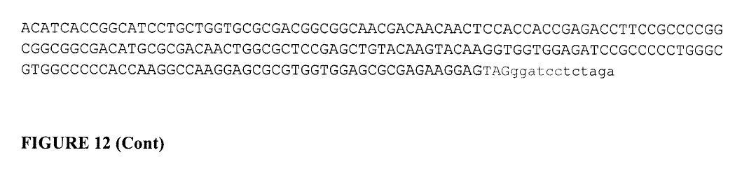

2. A recombinant CAP260, CAP174 or Ko224 HIV-1 Envelope protein of FIG. 6 (SEQ ID NOs: 55-60), or FIG. 12, or a combination thereof.

3. The recombinant protein of claim 2, wherein the protein is expressed as a gp160, gp140 or gp120.

4. A nucleic acid encoding any one of the proteins of claim 2.

5. A vector comprising the nucleic acid of claim 4.

6. The vector of claim 5, wherein the vector is suitable for use in a vaccine formulation or recombinant protein expression.

7. The vector of claim 6, where the vector is a pox virus vector.

8. A composition comprising the synthetic peptide of claim 1 or a combination thereof; the recombinant protein of claim 2 or a combination thereof; the nucleic acid of claim 4 or a combination thereof; or the vector of claim 5 or a combination thereof.

9. The composition of claim 8 comprising a trivalent combination of the CAP260, CAP174 and Ko224 peptides of claim 1.

10. The composition of claim 8 comprising a trivalent combination of the CAP260, CAP174 and Ko224 proteins of claim 2, or the nucleic acids of claim 4.

11. The composition of claim 10 wherein the protein is present as a monomer.

12. The composition of claim 8, wherein the composition is immunogenic.

13. The composition of claim 8, wherein the composition comprises an adjuvant.

14. A method of inducing antibodies in a subject comprising administering to the subject any one of the compositions of claim 8.

15. The method of claim 14, wherein the composition of claim 8 is administered as a boost in an immunization regimen following administration of ZM-96gp120 as a prime, and TV-1 and 1086C gp120 envelope proteins as a boost.

16. The method of claim 15, wherein the composition of claim 8 is administered as a multiple boost.

17. The method of claim 14, wherein the composition of claim 8 is administered as a prime in an immunization regimen along with administration of ZM-96gp120 as a prime, and TV-1 and 1086C gp120 envelope proteins as a boost.

18. The composition of claim 8, wherein the composition further comprises TV1 gp120 envelope and 1086C gp120 envelope.
